(19) United States
(12) Patent Application Publication
(10) Pub. No.: US 2003/0124547 A1
(43) Pub. Date: Jul. 3, 2003

(54) Hybridization assays for gene dosage analysis

(76) Inventors: Risa Peoples, Palo Alto, CA (US); Reuel Van Atta, Mountain View, CA (US)

Correspondence Address: Todd A. Lorenz, Esq., FLEHR HOHBACH TEST ALBRITTON & HERBERT LLP, Suite 3400, Four Embarcadero Center, San Francisco, CA, 94111-4187, US

(21) Appl. No.: 10/093,626

(22) Filed: Mar. 8, 2002

Related U.S. Application Data
(63) Continuation-in-part of application No. 09/390,124, filed on Sep. 3, 1999, now Pat. No. 6,495,676.
Application No. 09/390,124 is a continuation of application No. 09/149,161, filed on Sep. 4, 1998, now Pat. No. 6,277,570.

(51) Int. Cl. C12Q 1/68; C07H 21/04

(52) U.S. Cl. 435/6; 536/23.1

(57) ABSTRACT

Methods and compositions are provided for determining the dosage of target nucleic acid sequences. Probes comprising a crosslinking agent are combined with a sample that may comprise a target sequence that is complementary to a probe. Hybridization is allowed to occur between complementary sequences. The crosslinking agent is activated. Covalent bonds are formed between a probe and a target sequence if they are hybridized to each other, and a high-stringency wash step is employed to significantly lower background contamination. The crosslinked nucleic acids can then be detected and the resulting signal compared against a known diploid locus to determine the dosage of the target sequence. Dosage detection may be combined with the detection of polymorphisms, such as single nucleotide polymorphisms, to provide a more complete genetic profile at a locus or loci of interest.

### RELATED APPLICATIONS

This application is a continuation-in-part of U.S. patent application Ser. No. 09/390,124, which is a continuation of U.S. patent application Ser. No. 09/149,161, now U.S. Pat. No. 6,277,570.

### TECHNICAL FIELD

The field of this invention is nucleic acid sequence detection, and more specifically, the detection of gene dosage abnormalities and other genetic polymorphisms in genes of interest.

### BACKGROUND

Genetic polymorphism, or variation in DNA sequences, is widespread throughout the human genome and is a main reason for individual variation in protein function, which can lead to disease conditions. There are already a substantial number of genes, which when mutated, are known to be associated with various diseases in humans, including cystic fibrosis, Huntington's disease, beta thalassemia, sickle cell anemia, and the like. In some instances, such as with sickle cell anemia, there is a common point mutation in one gene that is associated with the disease. In other cases, such as with cystic fibrosis, there are numerous point mutations spread throughout the genes that are associated with the disease.

In addition to the identification of disease-associated genes, there is also increasing interest in pharmacogenetics, which attempts to harness rapidly-expanding genetic knowledge to identify genes involved in variable responses to drugs. Drug responsiveness can range from subtherapeutic and ineffective dosing at one extreme to toxic and potentially lethal overdosing at the other, and there is a growing body of evidence indicating that this variation may be due at least in part to genetic factors, including polymorphisms within one or more genes coding for protein products involved in critical metabolic and/or physiological pathways relevant for drug action. Understanding the genetic basis for the pharmacokinetic and pharmacodynamic factors involved in drug variability among individuals will enable truly personalized medicine, in which patients can be screened in advance to ensure proper drug selection and dosing based on their own genetic makeup, for maximum responsiveness and minimal toxicity.

Present experience with disease gene mutation identification suggests that gene dosage aberrations—deletions and/or duplications of large regions of DNA leading to an abnormal gene copy number—represent a significant proportion of phenotypically relevant genetic mutations. Although single nucleotide substitutions account for the majority of described polymorphisms to date, dosage abnormalities are likely to play an important role as well, and the detection of dosage abnormalities along with point mutations will likely be necessary for complete genotyping at many clinically important loci.

The current gold standard for clinical diagnosis of dosage abnormalities is fluorescent in situ hybridization (FISH), a method requiring expensive reagents, significant time to perform and the participation of highly skilled personnel. Other methods such as Southern blotting with densitometric analysis are even more involved. Current polymerase chain reaction (PCR)-based methods are primarily employed for recognizing 10-fold or greater differences in copy number; they cannot accurately discriminate 1- vs. 2-fold differences in gene copy number and are not amenable to large-scale gene dosage detection. Methods such as hot-stop PCR, for example, which claims to allow PCR target quantification, cannot provide two-fold copy discrimination. Similarly, quantitative fluorescent PCR, which employs PCR amplification with fluorescently-tagged primers to intradeletion and extradeletion sequences and software analysis of logarithmic-phase amplicon accumulation via signal intensity comparisons, is cumbersome for large-scale determinations. PCR amplification techniques in general also suffer from well-established complications, including enzyme inhibition, false priming, and asymmetric amplification of alleles, making their general utility for clinical diagnostics doubtful.

Moreover, conventional screening methods for point mutations also utilize PCR-based methods, and the assays typically employed do not provide for parallel screening of gene copy number and point mutations. This limitation arises because the detection of point mutations by hybridization-based assays requires the use of low-stringency wash conditions due to the low melting temperatures involved in forming complexes between an oligonucleotide probe and a target DNA differing by only a single nucleotide. The determination of gene dosage by comparison of hybridization signals between a test locus and an obligatory diploid copy locus requires high-stringency conditions. For signal intensity to correlate reliably to quantitative amounts of probe-target hybridization complexes, background hybridization levels must be both low (high signal-to-noise ratio) and reproducible. Traditionally, hybridization-based dosage assays have relied on probes of much longer length, capable of forming high melting temperature probe-target complexes that can withstand high-stringency washing. Thus, current hybridization methodology would make concomitant determination of gene dosage and point mutation in the same assay impossible.

What is needed is a hybridization technique providing accurate gene dosage determinations that is also amenable to large-scale gene dosage investigations. Also needed are improved hybridization techniques that provide for the parallel determination of gene dosage and point mutations in a single assay. The techniques must be capable of detecting a wide variety of mutational mechanisms within a single platform, including simple nucleotide substitutions; deletions and insertions of one to several base pairs; inversions; and large deletions and duplications of essentially indefinite size.

Relevant Literature

Articles that describe various techniques for detecting deletions and duplications include: Yau et al., J. Med. Genet. 1996, 33(7):550-558; Bentz et al., Genes Chromosomes Cancer 1998; 21(2):172-175; Geschwind et al., Dev. Genet. 1998; 23(3):215-229; Armour et al., Nucleic Acids Res. 2000; 28(2):605-609; Lindblad-Toh et al., Nat. Biotechnol. 2000; 18(9):1001-1005; Ruiz-Ponte et al., Clin. Chem. 2000; 46(10): 1574-1582; Jung et al., Clin. Chem. Lab. Med. 2000; 38(9):833-836; Kariyazono et al., Mol. Cell. Probes 2001; 15(2):71-73; Antonarakis, Nat. Genet. 2001; 27(3):230-232; Hodgson et al., Nat. Genet. 2001; 29(4):459-464.

Nucleic acid crosslinking probes for DNA/RNA diagnostics are disclosed in Wood et al., Clin. Chem. 1996; 42(S6):S196. Crosslinker-containing probes have been reported to be able to discriminate between single-base polymorphic sites in target sequences in solution-based hybridization assays. Zehnder et al., Clin. Chem. 1997; 43(9): 1703-1708.

### SUMMARY OF THE INVENTION

The present invention provides improved methods for detecting gene dosage abnormalities, either alone or in combination with other genetic polymorphisms of interest. In one embodiment, the invention provides a method for determining the copy number of a dosage region in a sample, comprising the steps of 1) hybridizing said dosage region to a first crosslinkable probe mixture, wherein said first crosslinkable probe mixture comprises at least one dosage reporter probe comprising a crosslinking agent, a label capable of producing a dosage signal and a sequence substantially complementary to at least a portion of said dosage region; 2) activating said crosslinking agent to form a first crosslinked nucleic acid complex, whereby a covalent crosslink occurs between said first crosslinkable probe mixture and said dosage region when said dosage region is present in said sample; 3) washing said first crosslinked nucleic acid complex at least once under high-stringency conditions; 4) detecting said dosage signal; and 5) determining the copy number of said dosage region based on the ratio of said dosage signal to a diploid signal.

Preferably, the diploid signal is obtained by the additional steps of hybridizing a second crosslinkable probe mixture to a diploid region in said sample and performing the activating, washing and detecting steps listed above to obtain said diploid signal; wherein said second crosslinkable probe mixture comprises at least one diploid reporter probe having a sequence complementary to at least a portion of said diploid region, a crosslinking agent and a detectable label capable of producing said diploid signal.

In a preferred embodiment, the first crosslinkable probe mixture further comprises at least one dosage capture probe, said dosage capture probe comprising a crosslinking agent, a label comprising a member of a specific binding pair and a sequence that is substantially complementary to at least a portion of said dosage region and is distinct from the sequence of said at least one dosage reporter probe. With this embodiment, one may separate said first crosslinked nucleic acid complex formed by said activating step using said capture probe. In a particularly preferred embodiment, the detectable label is a fluorophore and the member of a specific binding pair is biotin.

Preferably, the crosslinking agent is a photoactivatable crosslinking agent, and more preferably, the photoactivatable crosslinking agent is selected from the group comprising coumarin derivatives and aryl-olefin derivatives.

In another embodiment, the invention provides a method for determining the copy number of a dosage region in a sample, wherein said sample comprises at least one dosage region and at least one diploid region, comprising the steps of 1) hybridizing said at least one dosage region to a dosage probe mixture to form a dosage hybridization complex, said dosage probe mixture comprising at least one dosage reporter probe comprising a crosslinking agent, a label capable of producing a dosage signal and a sequence substantially complementary to at least a portion of said dosage region; 2) hybridizing said at least one diploid region to a diploid probe mixture to form a diploid hybridization complex, said diploid probe mixture comprising at least one diploid reporter probe comprising a crosslinking agent, a label capable of producing a diploid signal, and a sequence substantially complementary to at least a portion of said diploid region; 3) activating said crosslinking agent, whereby a covalent crosslink occurs between said diploid probe mixture and said diploid region to form a crosslinked diploid probe: diploid region complex, and between said dosage probe mixture and said dosage region to form a crosslinked dosage probe: dosage region complex when said dosage region is present in said sample; 4) washing said crosslinked dosage probe: dosage region complex and said diploid probe: diploid region complex at least once under high-stringency conditions; 5) detecting said dosage signal and said diploid signal; and 6) determining the copy number of said dosage region based on the ratio of said dosage signal to said diploid signal.

Preferably, the dosage probe mixture further comprises at least one dosage capture probe comprising a crosslinking agent, a label comprising a member of a specific binding pair, and a sequence which is substantially complementary to at least a portion of said dosage region and is distinct from the sequence of said at least one dosage reporter probe. With this embodiment, as noted above, one may separate the crosslinked dosage probe: dosage region complex formed by said activating step using said at least one dosage capture probe. Alternatively, or additionally, the diploid probe mixture may further comprise at least one diploid capture probe comprising a crosslinking agent, a label comprising a member of a specific binding pair, and a sequence that is substantially complementary to at least a portion of said diploid region and is distinct from the sequence of at said least one diploid reporter probe, such that one may separate the crosslinked diploid probe: diploid region complex formed by said activating step using said at least one diploid capture probe.

In a further embodiment, the invention provides a method for determining the copy number of a dosage region in a sample, comprising the steps of 1) hybridizing said dosage region to a dosage probe mixture, wherein said dosage probe mixture comprises a plurality of dosage probes comprising a crosslinking agent and having distinct sequences that are substantially complementary to a portion of said dosage region, said plurality of dosage probes further comprising: a) at least one dosage reporter probe comprising a label capable of producing a dosage signal; and b) at least one dosage capture probe comprising a label comprising a member of a specific binding pair; 2) activating said crosslinking agent to form a crosslinked dosage complex, whereby a covalent crosslink occurs between said plurality of dosage probes and said dosage region when said dosage region is present in said sample; 3) separating said crosslinked dosage complex formed by said activating step using said member of a specific binding pair; 4) washing said crosslinked dosage complex at least once under high-stringency conditions; 5) detecting said dosage signal; and 6) determining the copy number of said dosage region based on the ratio of said dosage signal to a diploid signal.

Preferably, this embodiment further comprises the additional steps of hybridizing a diploid probe mixture to a diploid region in said sample and performing said activating, separating, washing and detecting steps to obtain said diploid signal wherein said diploid probe mixture comprises: 1) at least one diploid reporter probe comprising a sequence complementary to at least a portion of said diploid region, a crosslinking agent and a label capable of producing said diploid signal, and 2) at least one diploid capture probe comprising a crosslinking agent, a label comprising a member of a specific binding pair, and a sequence that is substantially complementary to at least a portion of said diploid region and is distinct from the sequence of said at least one diploid reporter probe.

In a preferred embodiment, a method for determining the copy number of a dosage region in a sample is provided, wherein said sample comprises at least one dosage region and at least one diploid region, comprising the steps of: 1) hybridizing said at least one dosage region to a dosage probe mixture to form a dosage hybridization complex, said dosage probe mixture comprising a plurality of dosage probes comprising a crosslinking agent and having distinct sequences that are substantially complementary to a portion of said dosage region, and wherein at least one of said plurality of dosage probes further comprises a label capable of producing a dosage signal and at least one of said plurality of dosage probes further comprises a label comprising a member of a specific binding pair; 2) hybridizing said at least one diploid region to a diploid probe mixture to form a diploid hybridization complex, said diploid probe mixture comprising a plurality of diploid probes comprising a crosslinking agent and having distinct sequences that are substantially complementary to a portion of said diploid region, and wherein at least one of said plurality of diploid probes further comprises a label capable of producing a detectable diploid signal and at least one of said plurality of diploid probes further comprises a label comprising a member of a specific binding pair; 3) activating said crosslinking agent, whereby a covalent crosslink occurs between said diploid probe mixture and said diploid region to form a crosslinked diploid complex, and between said dosage probe mixture and said dosage region to form a crosslinked dosage complex when said dosage region is present in said sample; 4) separating said crosslinked dosage complex and said crosslinked diploid complex formed by said activating step using said member of a specific binding pair; 5) washing said crosslinked dosage complex and said crosslinked diploid complex at least once under high-stringency conditions; 6) detecting said dosage signal and said diploid signal; and 7) determining the copy number of said dosage region based on the ratio of said dosage signal to said diploid signal.

In a particularly preferred embodiment, a method for genotyping a target sequence in a sample is provided, wherein said target sequence comprises a dosage region and an interrogation region comprising an interrogation position, the method comprising the steps of: 1) hybridizing said dosage region to a first crosslinkable probe mixture to form at least one first hybridization complex, said first crosslinkable probe mixture comprising at least one dosage reporter probe comprising a crosslinking agent, a label capable of producing a dosage signal and a sequence substantially complementary to at least a portion of said dosage region; 2) hybridizing said interrogation region to a second crosslinkable probe mixture to form at least one second hybridization complex, said second crosslinkable probe mixture comprising at least one allele-specific detection probe comprising a crosslinking agent, a label capable of producing an interrogation signal and a sequence substantially complementary to the sequence upstream and downstream of the interrogation position in said interrogation region; 3) activating said crosslinking agent, whereby said first hybridization complex becomes covalently crosslinked when said dosage region is present in said sample, and said second hybridization complex becomes covalently crosslinked when said detection position is perfectly complementary to said interrogation position; 4) washing said crosslinked first and second hybridization complexes at least once under high-stringency conditions; 5) detecting said dosage signal to determine the copy number of said dosage region and detecting said interrogation signal to determine the identity of said interrogation position. With this embodiment, the second crosslinkable probe mixture may further comprise a plurality of allele-specific capture probes having distinct sequences that differ at said detection position, thereby enabling discrimination of alleles.

In a further embodiment, a method is provided for determining the dosage of a target sequence comprising a dosage region in a sample, comprising the steps of: 1) hybridizing said dosage region to a first crosslinkable probe mixture to form a first hybridization complex, said first crosslinkable probe mixture comprising at least one reporter probe comprising a detection region, a crosslinking agent and a label capable of producing a dosage signal, wherein said detection region is substantially complementary to said dosage region; 2) activating said crosslinking agent, whereby a covalent crosslink occurs between said detection region and said dosage region when said dosage region is present in said sample; 3) washing said at least one hybridization complex at least once under high-stringency conditions; 4) detecting said dosage signal; and 5) determining the dosage of said target sequence based on the ratio of said dosage signal to a diploid signal.

One may perform the hybridization-based assays of the present invention under high-stringency conditions despite a wide range of melting temperature complexes that must be discriminated, thereby enabling the parallel determination of gene dosage and any number of additional mutations in a single assay. The assay comprises covalent binding of the crosslinkable oligonucleotide probes of the present invention to target DNA following hybridization and prior to high-stringency washing. Whereas a typical low melting temperature hybridization complex would not survive the wash steps, the covalently bound complex remains intact. The improved hybridization techniques described and claimed herein enable accurate determinations of gene dosage in combination with other genetic mutations, and are amenable for large-scale genomic research and clinical diagnostics.

### DESCRIPTION OF THE FIGURES

FIG. 1 is a diagram illustrating a crossover event that can occur during meiosis and lead to abnormal gene copy number.

### DETAILED DESCRIPTION OF THE INVENTION

The present invention provides compositions and methods for making gene dosage determinations. Herein, the term “gene dosage” refers to the quantitative determination of gene copy number present in an individual's genome. Because the normal human genome is diploid, the normal gene dosage for non-X-linked genes is two. Whole-gene and larger (microscopic and submicroscopic chromosomal) deletions and duplications (gene dosage or gene copy number of one and three or more, respectively) confer specific phenotypes, and their diagnosis is of critical clinical importance.

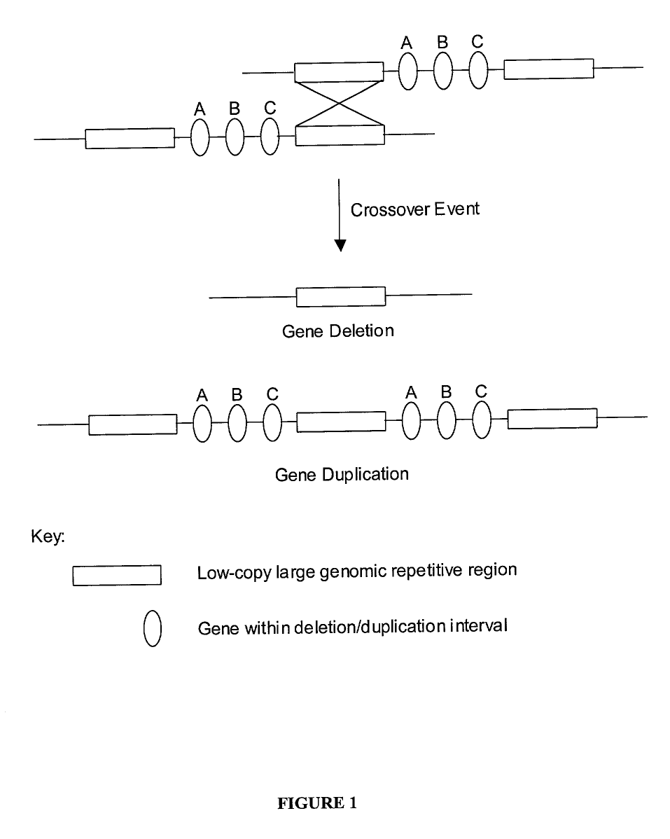

A schematic representation is provided in FIG. 1 demonstrating the kind of crossover event that can occur during meiosis and lead to abnormal gene copy number. As illustrated therein, a crossover event occurring at a low-copy large genomic repetitive region results in the duplication of genes A, B and C in one strand, and their deletion in the other strand. As described herein, the present invention provides methods and compositions for determining rapidly and accurately the gene copy number of genomic regions subject to these types of duplication and/or deletion events, referred to generally herein as “dosage regions.”

Following the methods of the present invention, one may also determine gene dosage in parallel with the detection of one or more additional types of polymorphism that may be present in a gene or genes of interest. The polymorphism may be either inherited or spontaneous, germiline or somatic, or a marker of interspecies variation. Polymorphisms or mutations of interest include those related to gene dosage abnormalities such as deletions and duplications, as well as substitutions, insertions, translocations, rearrangements, variable number of tandem repeats, short tandem repeats, retrotransposons such as Alu and long interspersed nuclear elements, single nucleotide polymorphisms (SNPs), and the like. By convention, sequence variants present at frequencies less than 1% are generally considered mutations, whereas those present at higher frequencies are considered polymorphisms. As used herein, the term “polymorphism” means any DNA sequence variation of any type or frequency.

In one embodiment, the present invention provides a method for determining the gene dosage in a sample for one or more genes of interest. Generally, the method comprises combining a probe mixture comprising at least a first set of crosslinkable capture and reporter probes with a sample comprising a target sequence, which may be present as a major component of the DNA from the target or as one member of a complex mixture. At least one target sequence is preferably provided in single-stranded form, and will comprise a dosage region and optionally one or more interrogation regions. The capture and reporter probes are characterized by having known sequences derived from the gene or genes of interest, with complementarity to the polymorphic sequences and diploid control locus as explained below. In one embodiment, the probes contain a photoactive coumarin crosslinking agent as described in U.S. Pat. No. 6,005,093, the disclosure of which is incorporated by reference herein. In an alternative embodiment, the probes contain a photoactive aryl-olefin crosslinking agent as described in U.S. Pat. No. 6,303,799, the disclosure of which is incorporated by reference herein.

In a preferred embodiment, the capture and reporter probes further comprise first and second detectable labels, respectively. The first detectable label of the capture probe preferably comprises a molecule, e.g., biotin, that can be captured on a solid support, whereas the second detectable label of the reporter probe preferably comprises a reporter molecule, e.g., a fluorophore, an antigen, or other binding-pair partner useful for direct or indirect detection methods. In a particularly preferred embodiment, the first detectable label allows for separation of the capture probe-target complexes, such as, e.g., a biotinylated probe exposed to streptavidin-coated beads, whereas the second detectable label provides for quantification of signal strength, such as, e.g., fluorescein.

The probe mixture and the target sequence comprising the region of interest are first allowed to hybridize under not greater than mild-stringency conditions. After sufficient time for hybridization has elapsed to form a detectable amount of double-stranded nucleic acid, the sample is then subjected to crosslinking conditions so that a covalent bond is formed between the region and any hybridized probes from the probe mixture. The sample is then assayed for detection of the first and second detectable labels, either concurrently or, more preferably, consecutively, as described herein.

In a preferred embodiment, the sample further comprises a diploid control locus, termed a “diploid region,” and a further probe mixture is provided comprising a second set of capture and reporter probes substantially complementary to the diploid region. In the detection step, the dosage signal generated by the crosslinkable reporter probes from the first set (directed to the dosage region of interest) is then compared against the diploid signal generated by the reporter probes from the second set (directed to the diploid region) to determine gene dosage accurately. Following the methods of the present invention, increased sensitivity and improved reproducibility can be obtained. Use of crosslinkable probes directed to gene dosage abnormalities such as deletion or duplication polymorphisms enables high-stringency washes of the hybridized probe-target complexes, which significantly lowers background contamination levels and improves the signal-to-noise ratio. Additional sets of capture and reporter probe combinations directed to other polymorphisms or mutations in the gene or genes of interest may also be employed concurrently in the same platform for the same clinical sample, thereby providing a more complete genetic profile of a given locus or loci in parallel with a dosage determination.

As will be appreciated by those in the art, the sample may comprise any number of things, including, but not limited to, bodily fluids (including, but not limited to, blood, urine, serum, lymph, saliva, anal and vaginal secretions, perspiration, and semen) or solid tissue samples of virtually any organism, with mammalian samples being preferred and human samples being particularly preferred; environmental samples (including, but not limited to, air, agricultural, water and soil samples); biological warfare agent samples; research samples; purified samples, such as purified genomic DNA, RNA, etc.; raw samples, such as bacteria, virus, genomic DNA, mRNA, etc. As will be appreciated by those in the art, virtually any experimental manipulation may have been done on the sample.

By “nucleic acid” or “oligonucleotide” or grammatical equivalents herein means at least two nucleotides covalently linked together. As will be appreciated by those of skill in the art, various modifications of the sugar-phosphate backbone may be done to facilitate the addition of labels or to increase the stability and half-life of such molecules in physiological environments. The nucleic acid may be single-stranded or double-stranded, as specified, or contain portions of both double-stranded and single-stranded sequence. The nucleic acid may be DNA, both genomic and cDNA, RNA, or a hybrid, where the nucleic acid contains any combination of deoxyribo- and ribo-nucleotides, and any combination of bases, including uracil (U), adenine (A), thymine (T), cytosine (C), guanine (G), inosine, xathanine hypoxathanine, isocytosine, isoguanine, etc. As used herein, the term “nucleotide” includes nucleotides as well as nucleoside and nucleotide analogs, and modified nucleotides such as labeled nucleotides. In addition, “nucleotide” includes non-naturally occurring analog structures. Thus, for example, the individual units of a peptide nucleic acid (PNA), each containing a base, are referred to herein as a nucleotide. The term “nucleotide” also encompasses locked nucleic acids (LNAs). Braasch and Corey, Chem. Biol. 2001; 8(1):1-7. Similarly, the term “nucleotide” (sometimes abbreviated herein as “NTP”), includes both ribonucleic acid and deoxyribonucleic acid (sometimes abbreviated herein as “dNTP”).

The compositions and methods of the invention are directed to the detection, quantification and/or genotyping of target sequences. The term “target sequence” or “target nucleic acid” or grammatical equivalents herein means a nucleic acid sequence on a single strand of nucleic acid. In a preferred embodiment, the target sequence comprises a dosage region. In another embodiment, the target sequence further comprises an additional polymorphism of interest, e.g., a SNP. Alternatively, the sample may comprise a plurality of distinct target sequences, each having one or more polymorphisms of interest. By “plurality” as used herein is meant at least two.

The target nucleic acid may come from any source, either prokaryotic or eukaryotic, usually eukaryotic. The source may be the genome of the host, plasmid DNA, viral DNA, where the virus may be naturally occurring or serving as a vector for DNA from a different source, a PCR amplification product, or the like. The target DNA may be a particular allele of a mammalian host, an MHC allele, a sequence coding for an enzyme isoform, a particular gene or strain of a unicellular organism, or the like. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of a genotyping or amplification reaction such as a ligated circularized probe, an amplicon from an amplification reaction such as PCR, etc. Thus, for example, a target sequence from a sample is amplified to produce a secondary target (amplicon) that is detected. Alternatively, what may be amplified is the probe sequence, although this is not generally preferred. Thus, as will be appreciated by those in the art, the complementary target sequence may take many forms. For example, it may be contained within a larger nucleic acid sequence, i.e., all or part of a gene or mRNA, a restriction fragment of a cloning vector or genomic DNA, among others. As is outlined more fully below, probes are made to hybridize to target and control sequences to determine the presence, sequence, or quantity of a target sequence in a sample. Generally speaking, the term “target sequence” will be understood by those skilled in the art.

If required, the target sequence is prepared using known techniques. For example, the sample may be treated to lyse the cells, using known lysis buffers, sonication, electroporation, etc., with purification and amplification occurring as needed, as will be appreciated by those in the art. The sample may be a cellular lysate, isolated episomal element, e.g., YAC, plasmid, etc., virus, purified chromosomal fragments, cDNA generated by reverse transcriptase, amplification product, mRNA, etc. Depending upon the source, the nucleic acid may be freed of cellular debris, proteins, DNA (if RNA is of interest), RNA (if DNA is of interest), size selected, gel electrophoresed, restriction enzyme digested, sheared, fragmented by alkaline hydrolysis, or the like. Importantly, however, and unlike the prior art, the benefits of improved sensitivity and reproducibility may be obtained following the methods of the present invention even without such additional DNA purification steps.

The target sequence may be of any length, with the understanding that longer sequences are more specific. In one embodiment, the target nucleic acid is provided with an average size in the range of about 0.25 to 3 kilobases (kb). Nucleic acids of the desired length can be achieved, particularly with DNA, by restriction enzyme digestion, use of PCR and primers, boiling of high molecular weight DNA for a prescribed time, and the like. Desirably, at least about 80 mol %, usually at least about 90 mol % of the target sequence, will have the same size. For restriction enzyme digestion, a frequently cutting enzyme may be employed, usually an enzyme with a four-base recognition sequence, or a combination of restriction enzymes may be employed, where the DNA will be subject to complete digestion.

Preferably, double-stranded target nucleic acids are denatured to render them single-stranded so as to permit hybridization of the capture and reporter probes of the invention. A preferred embodiment utilizes a thermal step, generally by raising the temperature of the reaction to about 95° C. in an alkaline environment, although chemical denaturation techniques may also be used. Where chemical denaturation has occurred, normally the medium will then be neutralized to permit hybridization. Various media can be employed for neutralization, particularly using mild acids and buffers, such as acetic acid, citric acid, etc. The particular neutralization buffer employed is selected to provide the desired stringency for hybridization to occur during the subsequent incubation.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents that may be included in the assays. These reagents include salts, buffers, innocuous proteins (e.g., albumin), detergents, etc., that may be used to facilitate optimal hybridization and detection, and/or reduce non-specific interactions. Also reagents that otherwise improve the efficacy of the assay, such as protease inhibitors, nuclease inhibitors, anti-microbial agents, etc., may be used, depending on the sample preparation methods and purity of the target.

In a preferred embodiment, a method of determining gene dosage is provided, wherein the target sequence comprises at least a portion of a genomic sequence that is known to be subject to deletion or duplication events, generally referred to herein as the “dosage region.” The dosage region will generally comprise a plurality of nucleotides, and more preferably, a plurality of contiguous nucleotides. As used herein, the corresponding region in the probe sequence that hybridizes with the dosage region or other sequence of interest is termed the “detection region.” Probes designed to hybridize with a dosage region in a target sequence are also referred to herein as “dosage probes.”

In a particularly preferred embodiment, the above method further comprises the parallel detection of an additional polymorphism of interest, such as, e.g., a parallel genotyping reaction. As is more fully outlined below, an interrogation region having a position for which sequence information is desired, generally referred to herein as the “interrogation position,” may be detected using additional probe sets complementary to portions of the interrogation region as described herein. In one such embodiment, the interrogation position is a single nucleotide, although in some embodiments, it may comprise a plurality of nucleotides, either contiguous or separated by one or more nucleotides within the interrogation region. As used herein, the corresponding probe base that hybridizes with the interrogation position base in a hybridization complex is termed the “detection position.” In the case where the detection position is a single nucleotide, the NTP in the probe that has perfect complementarity to the detection position is called a “detection NTP.” Probes designed to hybridize with at least a portion of the interrogation region in a target sequence are generally referred to herein as “detection probes,” whereas the subset of such probes comprising a detection position is referred to herein as “allele-specific detection probes.”

The term “allele” refers to individual genes that occupy the same locus or position on homologous chromosomes. When a subject has two identical alleles of a gene, the subject is said to be “homozygous” for the gene. When a subject has two different alleles of a gene, the subject is said to be “heterozygous” for the gene. Alleles of a specific gene can differ from each other in a single nucleotide or several nucleotides, and can include substitutions, deletions and insertions of nucleotides. An allele of a gene can also be a form of a gene containing a mutation. A given allele can therefore be defined by a multitude of sequence variations, which are referred to herein as “allelic variants.”

“Mutation” is a relative term meant to indicate a difference in the identity of a base at a particular position, termed the “interrogation position” herein, between two sequences. In general, sequences that differ from the norm (referred to herein by the term “wild type”) are herein referred to by the term “mutant.” However, particularly in the case of SNPs, what sequence represents wild type my be difficult to determine as multiple alleles can be observed relatively frequently in the population, and thus what constitutes a mutant in this context requires the artificial adoption of one sequence as a standard (i.e., wild type).

The present invention provides both capture and reporter probes that hybridize to regions of interest within a target sequence or a plurality of target sequences as described herein. In general, probes of the present invention are designed to be complementary to dosage, diploid, and/or interrogation regions of target sequence(s) (either the target sequence of the sample or to other probe sequences), such that hybridization occurs between the target and the probes of the present invention. This complementarity need not be perfect; there may be any number of base-pair mismatches that will interfere with hybridization between the target sequence and the corresponding detection regions in the probes of the present invention. However, if the number of mutations is so great that hybridization cannot occur under even the least stringent of hybridization conditions, the sequence is not a complementary target sequence. Thus, by “substantially complementary” herein is meant that the probe sequences are sufficiently complementary to the corresponding region of the target sequence (e.g., dosage, diploid or interrogation region) to hybridize under the selected reaction conditions.

Hybridization generally depends on the ability of denatured DNA to anneal when complementary strands are present in an environment below their melting temperature. The higher the degree of desired complementarity between the probe sequence and the region of interest, the higher the relative temperature that can be used. As a result, it follows that higher relative temperatures would tend to make the reaction conditions more stringent, whereas lower temperatures less so. For additional details and explanation of stringency of hybridization reactions, see Current Protocols in Molecular Biology, Ausubel et al. (Eds.).

Generally, the length of the probe and its GC content will determine the thermal melting point (Tm) of the hybrid, and thus the hybridization conditions necessary for obtaining specific hybridization of the probe to the region of interest. These factors are well known to a person of skill in the art, and can also be tested experimentally. The Tm is the temperature (under defined ionic strength and pH) at which 50% of the target sequence hybridizes to a probe. An extensive guide to the hybridization of nucleic acids is found in Tijssen, Hybridization with Nucleic Acid Probes: Theory and Nucleic Acid Probes, Vol. 1, 1993. Generally, stringent conditions are selected to be about 5° C. lower than the Tm for the specific sequence at a defined ionic strength and pH. Highly stringent conditions are selected to be greater than or equal to the Tm point for a particular probe.

Sometimes the term “dissociation temperature” (“Td”) is used to define the temperature at which half of the probe is dissociated from a target nucleic acid. In any case, a variety of techniques for estimating the Tm or Td are available, and generally described in Tijssen, supra. Typically, G-C base pairs in a duplex are estimated to contribute about 3° C. to the Tm, whereas A-T base pairs are estimated to contribute about 2° C., up to a theoretical combined maximum of about 80-100° C. However, more sophisticated models of Tm and Td are available and appropriate in which G-C stacking interactions, solvent effects, and the like are taken into account. For example, probes can be designed to have a desired dissociation temperature by using the formula: Td=(((((3×#GC)+(2×#AT))×37)−562)/#bp)−5; where #GC, #AT, and #bp are the number of G-C base pairs, the number of A-T base pairs, and the number of total base pairs, respectively, involved in the annealing of the probe to the template DNA.

The stability difference between a perfectly matched duplex and a mismatched duplex, particularly if the mismatch is only a single base, can be quite small, corresponding to a difference in Tm between the two of as little as 0.5° C. Tibanyenda et al., Eur. J. Biochem. 1984; 139(1): 19-27 and Ebel et al., Biochemistry 1992; 31(48):12083-12086. More importantly, it is understood that as the length of the complementary region increases, the effect of a single-base mismatch on overall duplex stability decreases. Thus, where there is a likelihood of unknown mismatches between the probe sequence and the target sequence, it may be advisable to include a longer complementary region in the probe, if the formation of a hybridized duplex is desired. Alternatively, where one is probing a known interrogation position with a plurality of allele-specific detection probes, it may be advisable to include a shorter complementary region in the probes to improve discrimination.

Thus, the specificity and selectivity of the probe can be adjusted by choosing proper lengths for the complementary regions and appropriate hybridization conditions. When the sample is genomic DNA, e.g., mammalian genomic DNA, the selectivity of the probe sequences must be high enough to identify the correct sequence in order to allow processing directly from genomic DNA. However, in situations in which a portion of the genomic DNA is first isolated from the rest of the DNA, e.g., by separating one or more chromosomes from the rest of the chromosomes, the selectivity or specificity of the probe may become less important.

The length of the probe, and therefore the hybridization conditions, will also depend on whether a single probe is hybridized to the target sequence, or several probes. In a preferred embodiment, several probes are used and all the probes are hybridized simultaneously to the target sequence. With this embodiment, it is desirable to design the probe sequences such that their Tm or Td is similar, such that all the probes will hybridize specifically to the target sequence. These conditions can be determined by a person of skill in the art by taking into consideration the factors discussed above.

A variety of hybridization conditions may be used in the present invention, including high-, moderate-, and low-stringency conditions; see, e.g., Sambrook et al., Molecular Cloning: A Laboratory Manual, 2nd ed., 1989 and Short Protocols in Molecular Biology, Ausubel et al (Eds.), 1992, hereby incorporated by reference. Stringent conditions are sequence-dependent and will differ depending on specific circumstances. Longer sequences hybridize more specifically at higher temperatures. Stringent conditions will be those in which the salt concentration is less than about 1.0 M sodiumion, typically about 0.01 to 1.0 M sodium ion concentration (or other salts) at pH 7.0 to 8.3, and the temperature is at least about 30° C. for short probes (e.g., 10 to 50 nucleotides (nt)) and at least about 60° C. for long probes (e.g., greater than 50 nt) in an entirely aqueous hybridization medium. Stringent conditions may also be achieved with the addition of helix destabilizing agents such as formamide. The hybridization conditions may also vary when a non-ionic backbone, e.g., PNA is used, as is known in the art.

Thus, the assays are generally run under stringency conditions that allow formation of the hybridization complex only in the presence of target and/or control. Stringency can be controlled by altering a step parameter that is a thermodynamic variable, including, but not limited to, temperature, formamide concentration, salt concentration, chaotrope concentration, pH, organic solvent concentration, etc. These parameters may also be used to control non-specific binding, as is generally outlined in U.S. Pat. No. 5,681,697. Thus it may be desirable to perform certain steps at higher stringency conditions to reduce non-specific binding, as described herein. The skilled artisan will recognize how to adjust the temperature, ionic strength, etc. as necessary to accommodate factors such as probe length and the like.

As will be appreciated by those in the art, the capture and reporter probes of the invention can take on a variety of configurations. The desired probe will have a sequence of at least about 10, more usually at least about 15, preferably at least about 16 or 17 and usually not more than about 1 kb, more usually not more than about 0.5 kb, preferably in the range of about 18 to 200 nt, and frequently not more than 50 nt, where the probe sequence is substantially complementary to the desired target sequence or control locus.

In one embodiment, particularly suited for gene dosage determination as described herein, the sequences of a first set of capture and/or reporter probes are selected to be substantially complementary to at least a portion of a known deletion or duplication region (termed a “dosage region”) in a gene or genes of interest. In this manner, the dosage region of interest in a given sample may be assayed for and quantified by comparing the resulting dosage signal against a diploid signal obtained from a known diploid locus in the sample, referred to herein as the “diploid region,” using a second set of probes substantially complementary to the diploid region.

Preferably, the diploid region is selected from a relatively unique region of the genome demonstrating minimal homology with other DNA, thereby minimizing the potential for cross-hybridizing sequence affecting signal strength. Sequence homology is easily ascertained through screening of the human genome through the sequence database maintained by the National Center for Biotechnology Information. As one of skill in the art is well aware, sequence from the non-pseudoautosomal X and Y chromosomal regions should be excluded as dosage varies with gender. Additionally, evidence for potential cell toxicity from over- or under-representation of gene dosage can also be inferred by an examination of chromosomal aberrations in cancer cells (Mitelman Database of Chromosome Aberrations in Cancer (2001). Mitelman F, Johansson B and Mertens F (Eds.), http://cgap.nci.nih.gov/Chromosomes/Mitelman). That is, cancer cells, having lost the normal controls over proliferation and DNA repair and being thus subject to the accumulation of mitotic errors, can indicate specific loci that are more likely to be cell-lethal when present in abnormal copy number. The scarcity of either deletions or duplications of a specific locus in tumor specimens can therefore be taken as evidence that the locus is toxic to cells in abnormal dosage and, therefore, will be reliably present in diploid copy number in the vast majority of human cells.

Selection of a diploid region in this manner is particularly suited to the development of assays for somatic dosage abnormalities in mixed-cell populations such as human tissues. Alternatively, so-called “housekeeping genes” can be selected as diploid controls. One of skill in the art will recognize these genes as those that have been identified as requisite for normal cell growth due to the provision by their product of an essential cell function. Because these genes are unlikely to be present in other than diploid copy, they represent good candidates to serve as diploid loci.

A number of different crosslinkable probes, as described in the examples below, can be included in the same probe mixture. For example, the probe mixture may include two or more probes directed to the same dosage region of interest but having distinct probe complementary sequences. With this embodiment one may guard against the possibility of unknown or rare, undefined SNPs significantly altering the efficacy of hybridization. In a further embodiment, additional probe sets are designed to detect additional polymorphisms of interest such as, e.g., one including a known SNP or other polymorphism, with one or more allele-specific detection probes having sequences substantially complementary to the interrogation region upstream and downstream of an interrogation position for which sequence information is desired, but differing in the corresponding interrogation NTPs. In this embodiment, the detection probe sequences are substantially complementary to the sequence surrounding the SNP at the interrogation position, but differ at the corresponding interrogation position with respect to the wild-type and mutant sequences, thereby enabling discrimination between normal and mutant genotypes, as described herein.

The probe sequence that binds to the target will usually be composed of naturally occurring nucleotides, but in some instances the sugar-phosphate chain may be modified, by using unnatural sugars, by substituting oxygens of the phosphate with sulfur, carbon, nitrogen, or the like, by modification of the bases, or absence of a base, or other modification that can provide for synthetic advantages, stability under the conditions of the assay, resistance to enzymatic degradation, etc. In one embodiment, modified nucleotides are incorporated into the probes that do not affect the Tms.

In addition, the probe may further comprise one or more labels (including ligand), such as a radiolabel, fluorophore, chemilumiphore, fluorogenic substrate, chemilumigenic substrate, biotin, antigen, enzyme, photocatalyst, redox catalyst, electroactive moiety, a member of a specific binding pair, or the like, that allow for capture or detection of the crosslinked probe. The label may be bonded to any convenient nucleotide in the probe chain so long as it does not interfere with the hybridization between the probe and the target sequence. Labels will generally be small, usually from about 100 to 1,000 Da. The labels may be any detectable entity, where the label is detected directly or by binding to a receptor, which in turn is labeled with a molecule that is readily detectable. Molecules that provide for detection in electrophoresis include radiolabels, e.g., ³²P, ³⁵S, etc., fluorescers, such as rhodamine, fluorescein, etc., ligand for receptors and antibodies, such as biotin for streptavidin, digoxigenin for anti-digoxigenin, etc., chemiluminescers, and the like. Alternatively, the label may be capable of providing a covalent attachment to a solid support such as bead, plate, slide, or column of glass, ceramic, or plastic.

As indicated herein, the methods of the present invention utilize crosslinkable probe mixtures directed to the dosage region, control region and/or other polymorphic region. There are extensive methodologies for providing crosslinking upon hybridization between the probe and the target to form a covalent bond. Conditions for activation may include photonic, thermal, and chemical, although photonic is the primary method, but may be used in combination with the other methods of activation. Therefore, photonic activation will be primarily discussed as the method of choice, but for completeness, alternative methods will be briefly mentioned.

The probes will have from 1 to 5 crosslinking agents, more usually from about 1 to 3 crosslinking agents. The crosslinking agents must be capable of forming a covalent crosslink between the probe and target sequence, and will be selected so as not to interfere with the hybridization. In a preferred embodiment, the crosslinking agents in the probe will be positioned across from a T, C, or U base in the target sequence.

For the most part, the compounds that are employed for crosslinking will be photoactivatable compounds that can form covalent bonds with a base, particularly a pyrimidine. These compounds will include functional moieties, such as coumarin, as present in substituted coumarins, furocoumarin, isocoumarin, bis-coumarin, psoralen, etc.; quinones, pyrones, α,β-unsaturated acids; acid derivatives, e.g., esters; ketones; nitrites; azido compounds, etc. A large number of functionalities can be generated photochemically and can form a covalent bond with almost any organic moiety. These groups include carbenes, nitrenes, ketenes, free radicals, etc. One can provide for a scavenging molecule in the bulk solution, normally excess non-target nucleic acid, so that probes that are not bound to a target sequence will react with the scavenging molecules to avoid non-specific crosslinking between probes and target sequences. Carbenes can be obtained from diazo compounds, such as diazonium salts, sulfonylhydrazone salts, or diaziranes. Ketenes are available from diazoketones or quinone diazides. Nitrenes are available from aryl azides, acyl azides, and azido compounds. For further information concerning photolytic generation of an unshared pair of electrons, see Schoenberg, Preparative Organic Photochemistry, 1968.

Another class of photoactive reactants are inorganic/organometallic compounds based on any of the d- or f-block transition metals. Photoexcitation induces the loss of a ligand from the metal to provide a vacant site available for substitutions. Suitable ligands include nucleotides. For further information regarding the photosubstitution of these compounds, see Geoffrey and Wrighton, Organometallic Photochemistry, 1979.

In one preferred embodiment, the crosslining agent comprises a coumarin derivative as described in co-pending U.S. patent application Ser. No. 09/390,124 and in U.S. Pat. No. 6,005,093, the disclosures of which are incorporated herein in their entirety. Briefly, with this embodiment the probes of the present invention benefit from having one or more photoactive coumarin derivatives attached to a stable, flexible, (poly)hydroxy hydrocarbon backbone unit. Suitable coumarin derivatives are derived from molecules having the basic coumarin ring system, such as the following: 1) coumarin and its simple derivatives; 2) psoralen and its derivatives, such as 8-methoxypsoralen or 5-methoxypsoralen (at least 40 other naturally occurring psoralens have been described in the literature and are useful in practicing the present invention); 3) cis-benzodipyrone and its derivatives; 4) trans-benzodipyrone and its derivatives; and 5) compounds containing fused coumarin-cinnoline ring systems. All of these molecules contain the necessary crosslinking group (an activated double bond) to crosslink with a nucleotide in the target strand.

Another preferred embodiment utilizes the aryl-olefm derivatives as the crosslinking agent, as described in U.S. patent application Ser. No. 09/189,294 and corresponding U.S. Pat. No. 6,303,799, the disclosures of which are incorporated herein in their entirety. In this embodiment, the aryl-olefin unit contains a photoactivated double bond that can covalently crosslink to suitable reactants in the complementary strand. Thus, the aryl-olefin unit can serve as a crosslinking moiety when attached via a linker to a suitable backbone moiety incorporated into the probe sequence.

The probes may be prepared by any convenient method, most conveniently synthetic procedures, where the crosslinker-modified nucleotide is introduced at the appropriate position stepwise during the synthesis. Alternatively, the crosslinking molecules may be introduced onto the probe through photochemical or chemical monoaddition. The above patent disclosures provide specific teachings regarding the incorporation of coumarin and aryl-olefin derivatives, which are incorporated by reference herein. Linking of various molecules to nucleotides is well known in the literature and does not require description here. See, for example, Oligonucleotides and Analogues: A Practical Approach, Echstein (Ed.), 1991.

The probe and target will be brought together in an appropriate medium and under conditions that provide for the desired stringency to provide an assay medium. Therefore, usually buffered solutions will be used, employing reagents, such as sodium citrate, sodium chloride, Tris, EDTA, EGTA, magnesium chloride, etc. See, for example, Sambrook et al., Molecular Cloning: A Laboratory Manual, 1988, for a list of various buffers and conditions, which is not an exhaustive list. Solvents may be water, formamide, DMF, DMSO, HMP, alkanols, and the like, individually or in combination, usually aqueous solvents. Temperatures may range from ambient to elevated temperatures, usually not exceeding about 100° C., more usually not exceeding about 90° C. Usually, the temperature for photochemical and chemical crosslinking will be in the range of about 20 to 70° C. For thermal crosslinking, the temperature will usually be in the range of about 70 to 120° C.

The amount of target nucleic acid in the assay medium will generally range from about 0.1 yoctomole to about 100 picomoles, more usually 1 yoctomole to 10 picomoles. The concentration of sample nucleic acid will vary widely depending on the nature of the sample. Concentrations of sample nucleic acid may vary from about 0.01 femtomolar to 1 micromolar. Similarly, the ratio of probe to target nucleic acid in the assay medium may vary, or be varied widely, depending upon the amount of target in the sample, the number and types of probes included in the probe mixture, the nature of the crosslinking agent, the detection methodology, the length of the complementarity region(s) between the probe(s) and the target, the differences in the nucleotides between the target and the probe(s), the proportion of the target nucleic acid to total nucleic acid, the desired amount of signal amplification, or the like. The probe(s) may be about at least equimolar to the target but are usually in substantial excess. Generally, the probe(s) will be in at least 10-fold excess, and may be in 10⁶-fold excess, usually not more than about 10¹²-fold excess, more usually not more than about 10⁹-fold excess in relation to the target. The ratio of capture probe(s) to reporter probe(s) in the probe mixture may also vary based on the same considerations.

Conveniently the stringency will employ a buffer composed of about 1× to 10×SSC or its equivalent. The solution may also contain a small amount of an innocuous protein, e.g., serum albumin, beta-globulin, etc., generally added to a concentration in the range of about 0.5 to 2.5%. DNA hybridization may occur at an elevated temperature, generally ranging from about 20 to 70° C., more usually from about 25 to 60° C. The incubation time may be varied widely, depending upon the nature of the sample, generally being at least about 5 minutes and not more than 6 hours, more usually at least about 10 minutes and not more than 2 hours.

After sufficient time for hybridization has elapsed, the crosslinking agent may be activated to provide crosslinking. The activation may involve illumination, heat, chemical reagent, or the like, and will occur through actuation of an activator, e.g., a means for introducing a chemical agent into the medium, a means for modulating the temperature of the medium, a means for irradiating the medium, and the like. If the activatable group is a photoactivatable group, the activator will be an irradiation means where the particular wavelength that is employed may vary from about 250 to 650 nm, more usually from about 300 to 450 nm. The illumination power will depend upon the particular reaction and may vary in the range of about 0.5 to 250 W. Activation may then be initiated immediately or after a short incubation period, usually less than 1 hour, more usually less than 0.5 hour. With photoactivation, usually extended periods of time will be involved with the activation, where incubation is also concurrent. The photoactivation time will usually be at least about 1 minute and not more than about 2 hours, more usually at least about 5 minutes and not more than about 1 hour.

The purpose of introducing covalent crosslinks between the probes and target DNA is to raise effectively the Tm of the complex above that attained by hydrogen bonding alone. This property allows wash steps to be performed at greater stringency than under initial hybridization conditions, thereby markedly reducing non-specific binding. Thus, the methods of the present invention provide hybridization complexes in which the probe(s) and target sequence(s) are covalently linked to one another, not just hydrogen bonded together. Therefore, harsher conditions that will disrupt any undesirable, nonspecific background binding, but will not break the covalent bond(s) linking the probe to its target sequence, may be employed. For example, washes with urea solutions or alkaline solutions could be used. Heat could also be used. The covalent linkage therefore allows for a significant improvement in the signal-to-noise ratio of the assay.

As described above, high-stringency conditions for the washing step generally employ low ionic strength and high temperature, or alternatively a denaturing agent, such as formanide. In a preferred embodiment, the wash conditions are 1× saline-sodium citrate (SSC), 0.1% Tween® 20 at room temperature (20-25° C.). In another preferred embodiment, the wash conditions are 50% formamide, 0.5% Tween® 20, 0.1×SSC at room temperature (20-25° C.).

After crosslinking of the hybridized probes in the probe mixture, the label(s) incorporated into the probe(s) may be detected. As noted above, a number of different labels that can be used with the crosslinkable probes are known in the art. For example, by having a label that is a member of a specific binding pair, e.g., antigen and antibody, such as digoxigenin and anti-digoxigenin; biotin and streptavidin; sugars and lectins; etc., one may separate the crosslinked nucleic acid on a solid support, e.g., container surface or bead, e.g., magnetic bead. By having a label that may provide a detectable signal, either indirect or direct, where the detectable label becomes crosslinked to the target nucleic acid, one has the opportunity to detect when said crosslinked nucleic acid has been separated onto a solid support or in some manner isolated. Labels may include fluorophores, chemiluminescers, radiolabels, and the like. For indirect detection, one will usually have a ligand that binds to a reciprocal member, which in turn is labeled with a detectable label. The detectable label may be any of the above labels, as well as an enzyme, where one can determine the presence of crosslinked probe by adding an enzyme substrate. Alternatively, this detectable label may serve as a member of another binding pair whose reciprocal pair generates a detectable signal, e.g., through the action of an enzyme on a substrate.

In one embodiment, one or more capture probes having as a label a member of a specific binding pair, e.g., biotin, are included in the probe mixture to achieve separation of the DNA sequence of interest from the remainder of the sample. In the preferred embodiment, the probe mixture comprises one or more reporter probes having a label that provides a detectable signal, and quantitative measurement may then be obtained by comparing the signals observed from the sample and a control. In a preferred embodiment described herein, the reporter probe is polyfluoresceinated to provide for increased signal generation. One may also use a substrate such as AttoPhos, as described herein, or other substrates that produce fluorescent products. With the present invention, the same sample can be contacted with different probe mixtures in different wells of the same microtiter plate in order to assay concurrently for gene dosage abnormalities such as deletions and duplications, and sequence differences such as SNPs. In an alternative embodiment, capture probes may be linked covalently to a solid support prior to performance of the assay.

Instead of separating the crosslinked probes(s)-target DNA from the assay medium, detection techniques can also be employed that allow for detection during the course of the assay. Alternatively, gel electrophoresis may be employed, and the amount of crosslinked probe to target determined by the presence of a radioactive label on the probe using autoradiography; by staining the nucleic acid and detecting the amount of dye that binds to the crosslinked probe; by employing an antibody that is specific for the crosslinked nucleic acid structures, particularly the crosslinked area, so that an immunoassay may be employed; or the like.

A diverse range of polymorphisms in one or more target sequences can be determined in parallel in accordance with the subject protocols. Clinical diagnostics is improved substantially with the present invention by the ability to assay simultaneously multiple mutational mechanisms of human genetic variation in a single platform, including both gene dosage and sequence abnormalities. The resulting genetic profile obtained for a given locus or loci will be more complete and can be used for risk profiling, chemopredictive testing, disease profiling, and pharmacogenetic testing, as well as for determining genetic mutations, genetic diseases, genotyping for trait analysis, and genotyping of other polymorphic sequences in humans, plants, and animals.

Specific genetic targets of interest include sequence variations such as SNPs. Generally, there may be a single nucleotide change in a single gene that is severe enough to cause disease in an individual (monogenic disease). However, many other genetic sequence variations are present that do not directly cause disease. These polymorphisms may, however, act in concert with one or several other genetic or environmental factors to produce a disease phenotype.

Moreover, the etiology of many monogenic conditions comprises a combination of single-to-several nucleotide mutations as well as large deletions and/or duplications. There are many current examples of pathologic conditions that can be caused by either a single-to-several nucleotide change amenable to PCR-based detection systems, or a large chromosomal rearrangent detectable by FISH or Southern blotting. Examples of large deletions as significant mutational events include familial breast and ovarian cancer due to BRCA1 mutation; Von Hippel-Lindau familial cancers; familial adenomatous polyposis coli colon cancers; and neurofibromatosis. Taken together, deletions can be expected to play a significant role in human variability at many loci. To date, no technology exists that can provide parallel assessment of SNPs and large deletions and/or duplications. Rather, an investigation is usually undertaken in a sequential fashion in which SNPs are examined first and large deletions examined separately. As described herein, the present invention can be used for the parallel assessment of SNP and dosage detection in one assay.

Clinical applications of the present invention include dosage testing of the following microdeletion syndromes:

TABLE 1

| | | Deletion | |
|---|---|---|---|
| Syndrome | Location | Size (Mb) | Prevalence |
| DiGeorge/Velocardiofacial | 22q11.2 | 3 | 1/4,000 |
| Williams | 7q11.23 | 1.7 | 1/20,000 |
| Smith-Magenis | 17p11.2 | 5 | 1/25,000 |
| HNPP | 17p11.2 | 1.5 | rare |
| Langer-Giedion | 8q24.1 | 5 | rare |
| Miller-Dieker | 17p13.3 | | rare |
| Alagille | 20p11.23-12.2 | | rare |
| Prader-Willi/Angelman | 15q11-13 | 4 | 1/20,000- |
| | | | 1/15,000 |
| Rubinstein-Taybi | 16p13.3 | | rare |
| WAGR | 11p13 | >0.7 | rare |
| Duchenne Muscular Dystro- | Xp21.2 | variable | 1/5,000 |
| phy | | | |
| Wolf-Hirschhorn | 4p16.3 | >5 | |
| Cri-du-chat | 5p15.2 | >5 | 1/50,000- |
| | | | 1/20,000 |
| Neurofibromatosis/DF | 17q11.2 | 1.5 | 1/40,000 |

Determination of gene dosage is of relevance in the diagnosis of duplications as well as deletions. Several clinical conditions are known to result from the presence of genes in triplicate or greater copy number as opposed to the normal two-copy state. Microduplication syndromes include the following:

TABLE 2

| | | Duplication | |
|---|---|---|---|
| Syndrome | Location | Size (Mb) | Prevalence |
| Charcot-Marie-Tooth 1A | 17p11.2 | 3 | 1/10,000 |
| Pelizaeus-Merzbacher | Xq22 | 0.8 | 1/300,000 |

Common whole-chromosome triploidies include involvement of chromosome 21 (Down syndrome), 13, and 18. As is true for gene deletions, there is also a need for accurate 3:2 gene dosage discrimination in clinical diagnostics.

For many of the syndromes described above, point mutations in single genes within the deletion/duplication region can have identical, or nearly identical, phenotypes. These syndromes include: HNPP/Charcot-Marie-Tooth 1A (PMP22); Pelizaeus-Merzbacher (PLP1); Angelman (UBE3A); Miller-Dieker (LIS1); Rubinstein-Taybi (CBP); Langer-Giedion (TRPS1 and EXT1); Duchenne and Becker muscular dystrophy (DMD); and Alagille (JAG1). Therefore, for many of these conditions, dosage testing and point mutation analysis need to be carried out either sequentially or in parallel.

In some cases, such as sickle cell anemia, there is a single common mutation. In other cases, such as cystic fibrosis, there are multiple mutations to be determined. By selecting the appropriate probe mixtures as described herein, one can detect simultaneously the presence or absence of all known mutations and polymorphisms in a gene or genes of interest, so that a more complete genetic profile may be obtained in a single assay.

Additional target sequences of interest include genes encoding for proteins involved in variable drug metabolism, such as CYP450 enzymes, e.g., 1A2, 2A6, 2C19, 2D6, 2E6, and 3A4, with human 2D6 and 2C19 particularly preferred. Additional sequences of interest include the mutation in sickle-cell anemia, the MHC associated with IDDM, mutations associated with cystic fibrosis, Huntington's disease, beta-thalassemia, Alzheimer's disease, and various cancers, such as those caused by activation of oncogenes (e.g., ras, src, myc, etc.) and/or inactivation of tumor suppressants (e.g., p53, RB, etc.).

In human cancers, for example, loss of expression of tumor suppressor genes is regularly associated with cancer progression. Deletions, loss of entire chromosomes, and methylation of CpG islands leading to repression of transcription are all common somatic mutations found in tumor tissues. Specific genes frequently lost in cancer cells include the following:

TABLE 3

| Gene | Cancer Type |
|---|---|
| hMLH1 | Colorectal, gastric |
| p14ARF and | Colorectal, melanoma, ovarian, lung, glioblastoma |
| p16INK4a | |
| CDKN2B | Hematologic malignancy |
| vHL | Renal |
| RB1 | Retinoblastoma |
| p53 | Lung |
| E-cadherin | Esophageal |
| GSTP1 | Prostate |
| RARbeta2 | Prostate |
| FHIT | Lung, breast |
| p73 | Acute lymphoblastic leukemia |

A second common mutational mechanism underlying the cellular transformation process is the amplification or serial duplication of oncogenes. These genes encode proteins whose overexpression or activation through point mutations causing constitutive expression, altered ligand affinity, or modified kinetics contributes to the malignant phenotype. Examples of specific genes amplified in cancer cells include the following:

TABLE 4

| Gene | Cancer Type |
|---|---|
| n-myc | Neuroblastoma |
| HER2 | Breast |
| cyclinD1 | Head and neck squamous cell carcinomas |
| c-myc | Gastrointestinal, bladder |

Comprehensive tumor genotyping will require methods that can assess deletions, duplications, and point mutations of genes in parallel. Kashiwagi and Uchida, Hum. Cell 2000; 13(3):135-341. Thus, each of the foregoing genes represents a target sequence of interest for analysis using the methods and compositions of the present invention.

Additional human genetic targets of interest include the genes encoding factor II, factor V, and the protein associated with hemochromatosis, all of which display genetic variations known to cause disease conditions.

Prothrombin (factor II) is the precursor to thrombin, which is a controlling factor in hemostatis and thrombosis. A genetic variation (G20210A) in the 3′ untranslated region of the prothrombin gene, is thought to affect negatively the regulation of gene expression, leading to increased risk for deep vein thrombosis. The genetic variation in the prothrombin gene is also associated with significantly increased risk for myocardial infarction when other risk factors are present, such as smoking and obesity.

The factor V Leiden mutation (G1691A) is the cause of 90% of the cases of individuals who display resistance to Activated Protein C (APC), which is the most common cause of inherited thrombophilia. This genetic mutation leads to the synthesis of a mutant factor V protein exhibiting decreased inactivation by APC.

Genetic hemochromatosis is an autosomal recessive disorder that causes an iron overload. Two mutations (G845A and C187G) in the common hereditary hemochromatosis (HFE) gene have been linked to significantly higher risk for an individual to develop hemochromatosis. The disease is characterized by high cellular iron levels that cause tissue damage, in particular in the liver, pancreas, joints, heart, and pituitary gland. Incidence of this disease is estimated to be 1 in 300 in the Northern European population.

Also of interest is determination of chromosome aneuploidies from fetal DNA obtained prenataly by amniocentesis, chorionic villus sampling, or other methods.

Kits are provided comprising probe mixtures capable of crosslinking as described previously. The probes are labeled to allow for easy detection of crosslinked nucleic acids. One may use radioactive labels, fluorescent labels, specific binding-pair member labels, and the like. The probes include sequences for hybridizing to a target sequence. In a preferred embodiment, the kit will comprise at least two probe sets directed to a dosage region of interest and a diploid control locus, respectively. As noted above, there may be a plurality of probe sets directed to additional target sequences to detect alternative polymorphisms that may be present in the gene or genes of interest. For example, pairs of probes may be used where the target sequence has a plurality of potential mutations spread through the gene. Ancillary materials may be provided, such as dyes, labeled antibodies, where a ligand is used as a label, labeled primers for use with PCR, and the like.

The following examples are offered by way of illustration and not by way of limitation.

### EXPERIMENTAL

### EXAMPLE 1

### Gene Dosage Assay for Deletion Mutation

The recurrent de novo deletion at chromosome 22q11 (del22q11) resulting in the endocrinologic, immunologic, cardiac, cognitive and psychiatric abnormalities of the velocardiofacial and DiGeorge syndromes, was used as a model system Deletions of this region are of variable size ranging from 1 to 3 megabases (mb), but all include a well-characterized “critical deletion region” of 1.2 mb common to the overwhelming majority of described cases. Morrow et al., Am. J Hum. Genet. 1995; 56(6): 1391-1403; Shaikh et al., Hum. Mol. Genet. 2000; 9(4):489-501.

Oligonucleotide Synthesis

An intragenic unique sequence from a region close to the N75 cosmid probe, currently in use in the FISH assay of the deletion region and well within the critical deletion region mapping to intronic sequence of the UFD1L gene, was used to design oligonucleotides for del22q11. An intronic sequence from the ANK2 gene at chromosome 4q25 was used to design oligonucleotide probes for use as the obligate two-copy control. Two capture probes, each of more than 35 bases (37 and 39 for 22q11; 38 and 39 for 4q25), were used for each assay to ensure that rare, undefined SNPs would not adversely affect hybridization.

Oligonucleotides were synthesized on an Expedite™ Nucleic Acid Synthesis System (PerSeptive Biosystems or Millipore) using standard DNA synthesis reagents. The capture probes contained a biotin molecule at the 3′-BiotinTEG-CPG (Glen Research), and the reporter probes were labeled at both the 3′ and 5′ ends with fluorescein (Fluorescein-CE phosphoramidite; Cruachem). Each probe type contained coumarin-based crosslinker nucleotides near the 3′ and 5′ terminus. To enable incorporation of the crosslinker into the oligonucleotides during automated synthesis, a fully protected phosphoramidite derived from 7-hydroxycoumarin, 1-O-(4,4′-dimethoxytrityl)-3-O-(7-coumarinyl)-2-O-(2-cyanoethyl-N,N-diisopropyl phosphoramidite) glycerol, was prepared.

The overall reporter probe concentrations in the two mixtures were adjusted so that control specimens gave roughly comparable signals from both the del22q11 and 4q25 control assays; 0.3 pmol and 0.2 pmol of each reporter was used in each assay for 22q11 and 4q25, respectively. For both assays, each capture probe was present at 0.5 pmol. Probe sequences are given below. Nucleotide positions are given for GenBank sequences for cosmid 102g9 (accession AC000068) for the 22q11 probes, and for BAC B240N9 (accession AC004057) for the 4q25 probes. For each probe, “X” denotes the coumarin-based crosslinker nucleotide, “F” denotes the fluorescein label, and “B” denotes the biotin label.

| | |
|---|---|
| Nu- | |
| cleotide | |
| Position | Sequence (5′ to 3′) |

22q11 Capture Probes

| | |
|---|---|
| 29069- | GAGCCTCCACTGACAXAGTCTCAGTTTTACTAGCAXAB |
| 29033 | |
| 29466- | TXACACAGACTGGTGGTTGGGCCTCTGACCATAACTGXAB |
| 29418 | |

22q11 Reporter Probes

| | |
|---|---|
| 29029- | FAXAAACCCTTGGTGGGTAGAXAF |
| 29008 | |
| 29510- | FAXAGGCAATAACTAAAGCCTAXAF |
| 29488 | |
| 28701- | FAXCAGCAGGACCCCTGGTAXAF |
| 28681 | |
| 28843- | FCCAGGATGGATTCCATTGCAXAF |
| 28822 | |
| 28960- | FAXAATGAAGAATGTTAXAGTACCTCTGGF |
| 28933 | |
| 28998- | FTGCAGGATCAXATCAGCTAACAXAF |
| 28975 | |
| 29151- | FCAGTACTTGGCCTGTGGCACAXAF |
| 29129 | |
| 29371- | FGGCTTCTCCTGGAGAXAGTAGGAAXAF |
| 29346 | |
| 29408- | FCGTGTTCCATCTCACCATTAGAXAF |
| 29385 | |
| 29533- | FTXAGCCAAGAATTTCAAGTAXAF |
| 29512 | |
| 29602- | FAXATGCTAATCTGATTATTTTAXAF |
| 29579 | |
| 29711- | FCXACTCCCAATTTTAATTTATTAXAF |
| 29687 | |
| 29760- | FAXAATGAACAAATGCTAAAGAXAF |
| 29738 | |
| 29791- | FAXCTCCATGGAGAAGCAAACATGTGXAF |
| 29765 | |
| 29891- | FAXACACCACTCAXAAGAACCTGATCF |
| 29867 | |
| 29115- | FAXATGGGCGTGGTTTTGGAXGF |
| 29095 | |
| 29343- | FCXAGGCTGCCTGATCCCTAGGTGGCXAF |
| 29317 | |
| 29864- | FAXTGAGTTTGCACTTTTATTCTCATTTCAXAF |
| 29834 | |
| 29989- | FAXATGTGTTCTAXAAGTAGTGGCTGF |
| 29965 | |
| 30017- | FAXATCAATTGCAXAGGTGTCAGTCF |
| 30040 | |

4q25 Capture Probes

| | |
|---|---|
| 22741- | CXAGGCAAACTCTCTAAATTAATGGTGTTTCCTCTAAXAB |
| 22779 | |
| 22311- | GGACTTGATTCTAGCAXAAAATGGGGAGCCACCATAXAB |
| 22348 | |

4q25 Reporter Probes

| | |
|---|---|
| 22017- | FAXATCTGCAAAGAACTAAGAGAXAF |
| 22050 | |
| 21934- | FAXAGATATTATTTAGCAGATTAXAF |
| 21957 | |
| 21978- | FGXACTTCATATCAGCAAGAGAGATCAXAF |
| 22005 | |
| 22046- | FAXATATATACTGAAAACAGCAXAF |
| 22068 | |
| 22098- | FAXACATGTAGCTACAGCAGGCTTAXAF |
| 22123 | |
| 22133- | FAXAATTTTATGGCATAGAXAF |
| 22152 | |
| 22156- | FAXAATCCATTTCCAGACCXAF |
| 22175 | |
| 22221- | FGXACTAGCTAAAGCAGTAAGAXAF |
| 22243 | |
| 22264- | FAXAATTGGAGTCTTCCTGCAXAF |
| 22285 | |
| 22361- | FAXAGGGTTATGATTAGTTTAXAF |
| 22382 | |
| 22409- | FAXACCCTGGACATGGGGTGACCCGXAF |
| 22434 | |
| 22611- | FGXACCAAAGGGATACAGTTGXAF |
| 22632 | |
| 22637- | FAXAATACATTGCATCATCTAXAF |
| 22658 | |
| 22700- | FAXACTCATAGCCTCTTCCCAGAXAF |
| 22723 | |
| 22789- | FTXAGTCAATCAACACATCAGGCAXAF |
| 22813 | |
| 22834- | FAXTGGGTTCTTATATTATGATGTGAXAF |
| 22860 | |
| 22868- | FAXAGCACAGCCAATAAGCTTTCAXAF |
| 22892 | |
| 22961- | FCXACTATTACACCTATTTTATXAF |
| 22983 | |
| 22987- | FAXATAATCATCTATATTTTATCXAF |
| 23010 | |
| 23091- | FAXACTTTTCAGGAGGTTCCTCTAXAF |
| 23115 | |

After synthesis, the probes were cleaved from the solid support and deprotected by incubating the support in concentrated ammonium hydroxide for 30 min at 55° C. The fully deprotected probes were purified via electrophoresis through denaturing polyacrylamide gels, followed by excision of the product bands and elution of the products. Zehnder and Benson, Am. J. Clin. Path. 1996; 106(1):107-111. The purified oligonucleotides were desalted by treatment through Sep-Pak® C18 cartridges (Waters).

Crosslinking Hybridization Assay Procedure

Sample preparation. Red blood cells were lysed by the addition of five volumes erythrocyte lysis buffer (1 mM EDTA, 10 mM KHCO₃, 155 mM NH₄Cl, pH 8.0) to one volume (1-3 mL) whole blood. Following 10 min incubation at room temperature, the samples were centrifuged at 750 g. The supernatant was decanted and the leukocyte pellet resuspended in 750 mL 1×SSC buffer (150 nM NaCl, 15 mM sodium citrate, pH 7.0). The cell suspension was transferred to a 2 mL microcentrifuge tube and centrifuged for 2 min at 3,500 g. The supernatant was discarded, 580 mL leukocyte lysis reagent (280 mM NaOH) added, and the cell pellet resuspended by vortexing. The resulting solution was used immediately or stored at −70° C. until required. The sample was heated in a boiling water bath for 5 min, vortexed to dissolve fully the cell debris, and then heated at 100° C. for an additional 20 min.

Assay setup and procedure. The gene dosage determination is based ultimately on the comparison of the fluorescent signals obtained from each sample after hybridization and crosslinking of the sample DNA to the intradeletion and extradeletion sets of probes. Aliquots of processed samples were placed into wells of a 96-well polypropylene microtiter plate (Coming Costar), along with negative (lysis buffer only) and normal (lysate from blood sample obtained from 22q11 diploid) controls. One of each probe mixture was added to each well under denaturing conditions.

Following addition of the probe reagents to the sample and control wells, 50 μL neutralization reagent (190 mM citric acid, 300 mM NaH₂PO₄, 1.5 M NaCl, 0.4% Tween® 20, 35% formamide) was added to each well. The loaded microplate was covered with a 2 mm thick Pyrex® filter and heated to 42° C. by placing it on a microplate heater that was positioned inside a UV crosslinking chamber 2.5 cm below UV lamps (UV-A bulbs, UVP Model CL1000-L; UVP). The samples were incubated for 20 min and then irradiated for 30 min at the same temperature. The flux delivered to the plate was approximately 30 mJ/cm². Following irradiation, the plate was removed from the heater and cooled to room temperature for 10 min. Next, 75 mg streptavidin-coated magnetic beads (Dynabeads® M-280 Streptavidin; Dynal) were added to each well to capture the crosslinked probe-target hybrids via the biotin moiety attached to the capture probes. Following a 30 min incubation at room temperature, the plate was placed over a set of bar magnets positioned between the wells such that the magnetic beads in each well formed a tight pellet along one side of the U-shaped well bottom.

After 30 seconds, the liquid in each well was removed by aspiration and the plate taken off the magnet assembly. The beads were then washed as follows: first with a pre-wash (0.1% SDS, 0.1×SSC, 0.001% Tween® 20), then with a gene dosage high-stringency wash (50% formamide, 0.5% Tween® 20, 0.1×SSC), and finally with a pre-incubation wash (1×SSC, 0.1% Tween® 20). The plate was placed onto the magnet assembly and the wash reagent removed at each step.

Immediately after washing the beads, 100 μL anti-fluorescein antibody-alkaline phosphatase conjugate (Boehringer Mannheim), diluted 1:3000 in 100 mM Tris-HCl (pH 7.5), 150 mM NaCl, 0.1% Tween® 20, 0.25% bovine serum albumin, was added to each well. The samples were incubated for 20 min at room temperature and then washed with 4×225 μL pre-incubation wash reagent using the procedure described above.

Upon completion of the final wash cycle, 100 μL of an alkaline phosphatase substrate (AttoPhos®; Promega), was added to each well and the plate incubated at 37° C. for 60 min. Finally, the fluorescent product produced from the reaction of AttoPhos® with alkaline phosphatase was detected by recording the fluorescence signal with a FluoroCount™ microplate reader (Packard Instrument).

Data Analysis

The deletion region dosage was determined from the ratio of the sample intradeletion (22q11) to extradeletion (4q25) signals corrected for background, as determined from the negative control readings. The sample signal ratios fell into one of two discrete intervals defined by comparison with normal controls: either the ratio of the sample signal to that of the normal control was close to one, consistent with the normal, two-copy dosage state, or it was close to 0.5, consistent with haploinsufficiency or deletion of one copy of the 22q11 region. The assay was developed using cell lines from a normal and a del22q11 individual and showed excellent discrimination.

Testing of Patient Samples

The 22q11 dosage status was determined on 45 patients in parallel results from FISH, the current gold standard for the evaluation of large deletions. Samples were obtained from Quest Cytogenetics, a large reference clinical laboratory. Samples were in the form of 1-3 mL aliquots of heparinized blood. Following performance of an assay, the ratio of intragenic to extragenic signal corrected for background was determined for each sample. Values between 0.8-1.2 were assigned a “non-deleted” status; whereas those between 0.4-0.6 were assigned a “deleted” status at 22q11. Results of the current assay correlated 100% with those obtained independently by FISH and are summarized below:

| | Crosslinking | Crosslinking Assay |
|---|---|---|
| | Assay “deleted” | “non-deleted” |
| FISH deleted | 2 | 0 |
| FISH non-deleted | 0 | 43 |

The data from the crosslinking assay demonstrates the ability of the methods of the present invention to assay gene copy number accurately. The assay allows the rapid diagnosis of large chromosomal microscopic and submicroscopic deletions. This technology represents a significant improvement over the current state-of-the-art, which requires many hours over several days of labor by highly skilled personnel. The present assay offers a substantially faster turnaround time for critical diagnostic information, as well as a reduction in performance time and complexity.

This assay is able to detect duplications as well as deletions accurately. The dosage ratios of the control and non-deleted cases showed a small variance around 1.0, which would easily allow discrimination of the 3:2 gene copy number expected in the case of duplications. Furthermore, the gene dosage assay is compatible with concurrent performance of SNP assays, as described herein. That is, the dosage and SNP detection assays can be performed concurrently in one 96-well plate. Currently, there is no other hybridization-based technology that can detect both mutational mechanisms in parallel. When combined with the gene dosage assay described above, multiple mutational mechanisms of human genetic variation may be assayed concurrently in a single platform.

### EXAMPLE 2

### Gene Dosage Assay for Duplication Mutation

Determination of gene dosage is relevant to the diagnosis of duplications as well as deletions. As is true for gene deletions, there is a need for accurate 3:2 gene dosage discrimination in clinical diagnostics. We used the previously described del22q11 assay on cell pellets obtained from two lymphoblast cell lines trisomic for the 22q11 region to establish the ability of this assay to detect gene duplications. Normalized ratios of 22q₁₁-to-diploid locus signals obtained from the two trisomic cell lines in two experiments averaged 1.48, with a standard deviation of 0.063 and a range of 1.41 to 1.56. This result demonstrates the ability of the present invention to detect gene duplications as well as deletions accurately.

### EXAMPLE 3

### Detection Assay for Two SNPs—Hereditary Hemochromatosis

Hereditary hemochromatosis (HH) is a common autosomal recessive disorder that is characterized by overabsorption of iron with consequent multiorgan failure secondary to iron overload. Edwards et al., N. Engl. J. Med. 1988; 318(21):1355-1362; McLaren et al., Blood 1995; 86(5):2021-2027. Because early diagnosis and therapy can prevent clinical complications entirely, HH presents a model system for presymptomatic detection at the molecular level. HFE, the disease causing gene of HH, encodes a 343 amino acid protein with high structural similarity to major histocompatibility complex class I molecules. Feder et al., Nat. Genet. 1996; 13(4):399-408; Camaschella and Piperno, Haematologica 1997; 82(1):77-84. The primary disease-causing mutation is a single G-to-A replacement at nucleotide position 845, encoding a protein, C282Y, with a cysteine-to-tyrosine amino acid substitution at residue 282. A second mutation of less clear significance is a C-to-G mutation at nucleotide position 187, leading to a protein, H63D, with a histidine-to-aspartate amino acid substitution at resideue 63. Homozygosity for the C282Y mutation and, in some cases, compound heterozygosity for the C282Y/H63D mutations confer the disease phenotype.

Current HH genotyping techniques include restriction fragment length polymorphism (RFLP) analysis and heteroduplex analysis, both of which are PCR-based. Jouanolle et al., Hum. Genet. 1997; 100(5-6):544-547; Jackson et al., Br. J. Hematol. 1997; 98(4):856-859. We have developed direct assays that do not require amplification of target DNA for the identification of the C282Y and H63D mutations. A set of two assays has been created for the two mutations (C282Y and H63D) to detect for mutant and wild-type alleles. Each assay utilizes two oligonucleotide probe sets that contain reporter probes complementary to the HFE gene and allele-specific capture probes. All oligonucleotide probes are modified with photoactivatable crosslinker molecules. A given assay contains two capture probes that are complementary to the sequence surrounding the mutation site, but differ at the mutation site with respect to mutant or wild type, thus enabling discrimination between the normal and mutant genotypes.

Sequences for the two capture probes, and the 14 and 16 reporter probes for the C282Y and H63D assay sets, respectively, are given below. The capture probes were biotinylated at the 3′-end as described in Zehnder et al., supra and are designated “CAP-WT” for the wild-type allele probe and “CAP-MUT” for the probe. The reporter probes were synthesized each containing two fluorescein groups either both at the 5′ terminus or one each at the 3′ and 5′ terminus. The coumarin-based crosslinking nucleotide (denoted “X”) was incorporated in place of a single nucleotide at one position within the sequence.

| | |
|---|---|
| Oligo ID | Sequence (5′-to-3′) |

C282Y Oligonucleotide Probe Sequences

| | |
|---|---|
| C282Y-REP1 | TGGCAAGGGTAAACAXATCCCC |
| C282Y-REP2 | ATCCTTCCTCTTTCCTGTCAXG |
| C282Y-REP3 | GCCTCCTTTGGTGAAGGTGACAXAT |
| C282Y-REP4 | GGGACCTACCAGGGCTGGATXA |
| C282Y-REP5 | TGGCTGTACCCCCTGGGGAAXA |
| C282Y-REP6 | AXAGCACCTACTATTTTGCAAG |
| C282Y-REP7 | GTCATTGGAGTCATCAGTGGAXT |
| C282Y-REP8 | GTCGTCATCTTGTTCATTGGAXT |
| C282Y-REP9 | GXAGCATTTTTCTCATTTATATTC |
| C282Y-REP10 | AXAGTCTCTAATTCAACAAACATC |
| C282Y-REP11 | AXAGCACCTACTATTTTGCAXG |
| C282Y-REP12 | GTGGCTATTCTCAGAACCCAXA |
| C282Y-REP13 | CCTAAAGCAGGCAGGAAGCAXA |
| C282Y-REP14 | AXTGAGAGCCAGGAGCTGAG |
| C282Y-CAP-WT | AXATACGTGCCAGGTG |
| C282Y-CAP-MUT | AXATACGTACCAGGTGG |

H63D Oligonucleotide Probe Sequences

| | |
|---|---|
| H63D-REP1 | AXACCCATGGAGTTCGGGGCTCC |
| H63D-REP2 | AXAATGTTAAGGTGTGTCTAGCC |
| H63D-REP3 | CATGATTAGACAGACTCCACTAXA |
| H63D-REP4 | GCCACCCCCTCACTGCCCCCAXA |
| H63D-REP5 | GGAGCAGGATGTTGAGGCTCCAXA |
| H63D-REP6 | GXACCTGGAGACAGAGCAACAGGC |
| H63D-REP7 | GCACCCATGAAGAGGTAGTGCAXA |
| H63D-REP8 | AACATGTGATCCCACCCTTTCAXA |
| H63D-REP9 | GTGATTTTCCATAATAGTCCAXA |
| H63D-REP10 | GXAGGTGAGGCCCCCTCTCCACAT |
| H63D-REP11 | AXAGATGAAAAGCTCTGACAACC |
| H63D-REP12 | GACTTCCAGCTGTTTCCTTCAAXA |
| H63D-REP13 | CCCTCTTCCCTGCTCCCACAAXA |
| H63D-REP14 | GACCAAATGATCTCAGGAAGCAXA |
| H63D-REP15 | CCAGCCTTGTTAACTGCAACCAXA |
| H63D-REP16 | TGCAGGGTGTGGGACTCTGGAXA |
| H63D-CAP-WT | GACTCTCATGATCATAXA |
| H63D-CAP-MUT | GACTCTCATCATCATAXA |

The assay was validated on a cohort of samples at the University of Cologne. Blood specimens were obtained from blood donors with informed consent under an institutional review board-approved protocol. All specimens were assayed with each probe set for the C282Y alleles and the genotype of each individual determined by comparison of the fluorescent signals obtained. Samples found to be heterozygous for C282Y were then tested for H63D to investigate potential C282Y/H63D heterozygosity. The blood samples were then assayed by a PCR-RFLP method and the results from both methods compared. The general protocol for both C282Y and H63D assays is identical.

Leukocytes isolated from blood samples using a red blood cell lysis procedure were resuspended in leukocyte lysis reagent (0.28 M NaOH). The mixture was either boiled at 100° C. for 30 min immediately prior to running an assay or stored at −20° C. for up to 14 days before boiling. For each assay, processed samples were placed into two wells of a 96-well polypropylene microtiter plate. Each assay plate contained four negative controls (unboiled leukocyte lysis reagent) and two positive controls (50 amol per well of a PCR amplicon covering the assay locus amplified from either a C282Y or H63D heterozygote in unboiled leukocyte lysis reagent). Two different probe solutions were prepared, each containing the same set of locus specific reporter probes and one of the two allele-specific capture probes. Aliquots of each probe solution were added to one of each sample well, as well as to two of the negative and one of the positive control wells. Subsequent neutralization of the solutions, photo crosslinking, and addition of the strepatavidin-coated magnetic beads are as described in Zehnder et al., supra. The beads were washed twice with a wash reagent (0.15 M NaCl, 0.015 M sodium citrate, 0.1% Tween® 20). The beads were then incubated in the presence of anti-fluorescein antibody-alkaline phosphatase conjugate (DAKO), washed four times and resuspended in a solution containing AttoPhos®. The fluorescence signal was determined by analyzing the plate with a FluoroCount™ microplate reader.

Genomic DNA was extracted from whole blood using the QIAamp® DNA Blood Midi Kit (QIAGEN). Sequences flanking the variant codons 282 and 63 were amplified by PCR, the amplicons digested with Rsa I (for C282Y) or Mbo I (for H63D) (Roche), size-fractionated by agarose gel electrophoresis, and then genotyped as described in Jouanolle et al., supra, and in Merryweather-Clarke et al., J. Med. Genet. 1997; 34(4):275-278.

Determining the genotype of an individual with the crosslinking assay is based on the relative signals obtained with the two allele-specific capture probe preparations. The net sample signal (NSS; sample signal adjusted for background by subtraction of the negative control signal) ratio is defined for each sample as the mutation NSS divided by the wild-type NSS. The NSS ratio intervals that define a particular genotype were set prior to testing the donor samples by assaying PCR amplicons derived from individuals known to be homozygous wild type, heterozygous, or homozygous mutant for both mutations. PCR-RFLP comparison testing was carried out on all samples.

The validation protocol was performed on a total of 1668 blood samples. Of these, 1510 were found to be homozygous wild-type at codon 282, 157, heterozygous for C282Y; and 1, homozygous for C282Y. The 157 heterozygotes were then assayed for the H63D mutation. Of these, 115 were homozygous for wild-type alleles at codon 63, and 25 were heterozygous, indicative of C282Y/H63D compound heterozygosity. No H63D homozygotes were identified. The results of these experiments were in complete agreement with those obtained by using the PCR-RFLP assay.

The crosslinking technology enables detection of the HFE G845A genetic (nucleotide) and C187G mutations without the laborious steps of DNA purification, PCR, and RFLP analysis, and eliminates problems due to sample inhibition and contamination often associated with nucleic acid amplification methods. A further advantage is the simultaneous processing of DNA samples in a large scale using the microtiter plate format. With automated detection, the crosslinking assay can be finished within four hours. Large-scale, presymptomatic screening of blood donors for the C282Y and H63D mutations should identify individuals at risk for HH, who are then candidates for prophylactic phlebotomy, which returns the life expectancy to the normal range. For such a screening regimen to be implemented, the genotyping tests need to be accurate, low-cost, and automatable. The crosslinking assay procedure described here demonstrates an efficient, simple, and rapid method of genotyping HFE mutations that, with automation, would be suitable for routine genetic analysis in a large-scale format.

### EXAMPLE 4

### Parallel Ascertainment of Hereditary Hemochromatosis HFE Genotype and 22q11 Gene Dosage

One strength of the crosslinking technology is that it allows target-probe complexes to withstand wash stringencies that are not only higher than in the absence of covalent crosslinking, but also equivalent over a broad range of hybridization-only Tm values. We have successfully performed the HH H63D and del22q11 gene dosage assays in parallel using a single microtiter plate, thereby demonstrating the ability of the present invention to assay SNP and dosage genotypes in a single platform.

A blood cell pellet isolated from an individual known to be homozygous wild type at codon 63 of the HFE gene and in possession of the common two copies of 22q11 was processed as described in Example 1. The H63D and del22q11 dosage assays were performed in parallel in separate wells of a single microtiter plate as described in Examples 3 and 1, respectively. Samples were assayed in duplicate for each assay along with controls for each assay as described previously. The experimental procedures are identical for both assays with the following exceptions: 1) Probe mixtures for the individual assays are as described in each example. Aside from the specific probe sequences, all reagents are otherwise of identical composition for both assays; 2) The high-stringency wash steps prior to incubation with the anti-fluorescein antibody-alkaline phophatase conjugate utilize different composition solutions for the two assays as described in Examples 1 and 3. The high-stringency wash step was therefore modified from both protocols. Following incubation of the crosslinked hybridization solution with magnetic beads, the captured target-probe complexes were washed once each with:

(1) 0.1% SDS, 0.1×SSC, 0.001% Tween® 20

(2) 20% formamide, 0.03×SSC, 0.5% Tween® 20

(3) 1×SSC, 0.1% Tween® 20

The beads were then resuspended in the anti-fluorescein antibody-alkaline phophatase conjugate reagent and further processed as stated for both HH H63D and del22q11 assays in Examples 3 and 1, respectively.

The following results were obtained: H63D A1 (wild-type capture probe mixture) (NSS) of 36.5; H63D A2 (mutant capture probe mixture) −4.5. The resulting negative ratio (−4.5:36.5) is indicative of a homozygous wild-type genotype. Meanwhile, the 22q11 NSS was 202.5, whereas that for 4q25 was 195, yielding a normalized NSS ratio of 1.07, consistent with normal diploid copy number at 22q11. These results demonstrate the ability of the present invention to assess multiple mutational mechanisms in parallel.

### EXAMPLE 5

### Polyfluoresceinated Probes

In a particularly preferred embodiment, multi-fluorescein probes are utilized in the subject methods to increase signal generation. DNA synthesis was performed on an Expedite™ Nucleic Acid Synthesis System (PerSeptive Biosystems or Millipore) DNA synthesizer using standard DNA synthesis reagents, except where otherwise stated. DMT-hexa-ethyloxy CED phosphoramidite (spacer-18) and Fmoc-amino-DMT C-3 phosphoramidite (amino-C3) were obtained from ChemGenes. 5(6)-Carboxyfluorescein-N-hydroxysuccinimide ester (FLUOS) was obtained from Roche. XL10 crosslinking phosphoramidite was synthesized as described in U.S. Pat. No. 6,005,093.

The first step in probe production was large-scale synthesis of the poly-amino-spacer 3′ tail. The synthesis proceeded with 40 couplings of amino-C3 phosphoramidite alternating with the spacer-18 phosphoramidite (80 total couplings) employing 1000 Å dT CPG in a 1.0 μmol-scale column with retention of the final trityl protecting group. The CPG was then transferred to a 0.2 μmol-scale column, and the DNA synthesis for each probe was completed with incorporation of the crosslinking phosphoramidite. Following synthesis, the CPG was placed overnight in concentrated ammonia (800 μL) at 45° C. to cleave the DNA from the solid support and to deprotect the DNA and the polyamine tail. The DNA-containing ammonia solution was evaporated for 10 min in a Speed Vac® (Savant), extracted once with butanol (1.0 mL), and then evaporated to dryness.

Probes were purified on a 6% denaturing polyacrylamide gel. The desired bands were excised from the gel, and the DNA was extracted by the crush-and-soak method. Desalting was performed using reverse-phase Sep-Pak® cartridges. This procedure involved activation of the column with 5 ml acetonitrite, followed by 10 mL water, loading of the sample, and then 10 mL of to desalt; and finally 1.0 mL of 60:40 methanol:water to elute the DNA. The DNA solution was evaporated to dryness using a Speed Vac® and then the resulting residue was redissolved in 110 μL water. DNA concentration was determined from the absorbance at 260 nm.

Fluorescein labeling was performed as follows: 1000 pmol of amino-spacer probe in 25 μL 100 mM NaHCO₃ (pH 8.5) were added to 25 μL of a DMSO solution containing 1.0 mg FLUOS and then incubated at 50° C. After one hour, the unreacted FLUOS reagent was separated from the fluorescein-labeled probe using a Centricon® YM-30 centrifugal filter (Millipore). Concentration was repeated five times with 2 mL of diluent added for each spin. The first two diluents were composed of 50:50 100 mM NaHCO₃ (pH 8.5):DMSO followed by one each of 100 mM NaHCO₃ (pH 8.5), water, and finally 10 mM Tris-HCl (pH 7.5). After the final spin the probe solution (˜30 μL) was diluted to 200 μL with 10 mM Tris-HCl (pH 7.5). The final concentration of the probe and the degree of fluorescein labeling were determined from the absorbance at 260 and 494 nm, respectively, (in 100 mM Na₂CO₃) in conjunction with calculated extinction coefficient of the DNA and the extinction coefficient of fluorescein (78,000 M⁻¹ cm⁻¹ at 494 nm). Typically, the degree of fluorescein incorporation was 20-25 per probe.

### EXAMPLE 6

### 15q11-13 Gene Dosage Assay

Deletion of one copy of the 15q11-13 region including the small nuclear ribonucleoprotein polypeptide N (SNRPN) gene is associated with the Prader-Willi and Angelman syndromes, thereby making its molecular diagnosis of clinical importance. Therefore, in a further embodiment, a 3′-SNRPN gene dosage assay was performed on lysed leukocyte pellets tor determine the 15q11 region copy number. The control locus assay at 4q25 developed for the 22q11 deletion assay described in Example 1 served the same role for this assay.

The reporter probe system was modified to use the polyfluoresceinated probes described in Example 5. The 3′-SNRPN and 4q25 assays contain 4 reporter probes each. All reporter probes were labeled with roughly 20-30 fluorescein units per oligonucleotide.

| | |
|---|---|
| Oligonucleotide Identification | Sequence (5′-to-3′) |

| | |
|---|---|
| 3′-SNRPN-CAPA | AXAAATATGAACTTAGACCCCCACCTAAXA |
| 3-′SNRPN-CAPB | AXAGCCTTTCTTTGCCTATTAGAATTGGATACATTAXA |
| 3′-SNRPN-REP1 | FAXATTTTTGCACACACCACTGGCCAXAF |
| 3′-SNRPN-REP2 | FAXATGCGCCATAACCACAXTF |
| 3′-SNRPN-REP3 | FAXAAGAAAATATCCCTAACTCTAXAF |
| 3′-SNRPN-REP4 | FAXATGTCTACCTGTTTTTTAAXAF |
| 4q25-CAPA | CXAGGCAAACTCTCTAAATTAATGGTGTTTCCTCTAAXA |
| 4q25-CAPB | GGACTTGATTCTAGCAXAAAATGGGGAGCCACCATAXA |
| 4q25-REP1 | AXAGGGTTATGATTAGTTTAXA |
| 4q25-REP2 | AXAATACATTGCATCATCTAXA |
| 4q25-REP3 | AXACTCATAGCCTCTTCCCAGAXA |
| 4q26-REP4 | AXTGGGTTCTTATATTATGATGTGAXA |

The 3′-SNRPN-to-4q25 NSS ratio (NSSR) was used to determine the overall 15q11 region copy number. All probe mixtures were prepared using a final amount of 0.5 pmol of each capture probe per well and 0.2 pmol of each reporter probe per well. The assay protocol is otherwise identical to that described in Example 1.

The assay was validated on lysed leukocyte cell pellets obtained from previously characterized lymphoblastoid cell lines created from a cohort of 14 15q11-13 deletion subjects (11 Prader-Willi and 3 Angelman syndrome subjects) and 11 normal control subjects. Diagnostic criteria were defined for normalized 3′-SNRPN-to-4q25 NSSR as follows: single copy (i.e., deletion) 0.35-0.65 (theoretical value of 0.5); double copy (i.e., normal state 0.85-1.15 (theoretical value of 1). Results are given below:

| | | | | Standard |
|---|---|---|---|---|
| Genotype | Number | Average NSSR | NSSR Range | Deviation |
| del15q11-13 | 12 | 0.544 | 0.44-0.65 | 0.069 |
| Normal Control | 11 | 1.02 | 0.93-1.13 | 0.067 |

All results fell within expected ranges and showed 100% concordance with results obtained from standard methods. These results demonstrate the accuracy of the assay for determining the 15q11-13 region copy number.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A method for determining the copy number of a dosage region in a sample, said method comprising:
a) hybridizing said dosage region to a first crosslinkable probe mixture, wherein said first crosslinkable probe mixture comprises at least one dosage reporter probe comprising a crosslinking agent, a detectable label capable of producing a dosage signal and a sequence substantially complementary to at least a portion of said dosage region;
b) activating said crosslinking agent to form a first crosslinked nucleic acid complex, whereby a covalent crosslink occurs between said first crosslinkable probe mixture and said dosage region when said dosage region is present in said sample;
c) washing said first crosslinked nucleic acid complex at least once under high-stringency conditions;
d) detecting said dosage signal; and
e) determining the copy number of said dosage region based on the ratio of said dosage signal to a diploid signal.

2. The method of claim 1, comprising the additional steps of hybridizing a second crosslinkable probe mixture to a diploid region in said sample and performing said activating, washing and detecting steps to obtain said diploid signal; wherein said second crosslinkable probe mixture comprises at least one diploid reporter probe having a sequence complementary to at least a portion of said diploid region, a crosslinking agent and a detectable label capable of producing said diploid signal.

3. The method of claim 1, wherein said first crosslinkable probe mixture further comprises at least one dosage capture probe, said dosage capture probe comprising a crosslinking agent, a label comprising a member of a specific binding pair and a sequence that is substantially complementary to at least a portion of said dosage region and is distinct from the sequence of said at least one dosage reporter probe.

4. The method of claim 3, comprising the additional step of separating said first crosslinked nucleic acid complex formed by said activating step using said capture probe.

5. The method of claim 3, wherein said detectable label is a fluorophore and said member of a specific binding pair is biotin.

6. The method of claim 1, wherein said crosslinking agent is a photoactivatable crosslinking agent.

7. The method of claim 6, wherein said photoactivatable crosslinking agent is selected from the group comprising coumarin derivatives and aryl-olefin derivatives.

8. A method for determining the copy number of a dosage region in a sample, wherein said sample comprises at least one dosage region and at least one diploid region, said method comprising:
a) hybridizing said at least one dosage region to a dosage probe mixture to form a dosage hybridization complex, said dosage probe mixture comprising at least one dosage reporter probe comprising a crosslinking agent, a detectable label capable of producing a dosage signal and a sequence substantially complementary to at least a portion of said dosage region;
b) hybridizing said at least one diploid region to a diploid probe mixture to form a diploid hybridization complex, said diploid probe mixture comprising at least one diploid reporter probe comprising a crosslinking agent, a detectable label capable of producing a diploid signal, and a sequence substantially complementary to at least a portion of said diploid region;
c) activating said crosslinking agent, whereby a covalent crosslink occurs between said diploid probe mixture and said diploid region to form a crosslinked diploid probe: diploid region complex, and between said dosage probe mixture and said dosage region to form a crosslinked dosage probe:dosage region complex when said dosage region is present in said sample;
d) washing said crosslinked dosage probe:dosage region complex and said diploid probe: diploid region complex at least once under high-stringency conditions;
e) detecting said dosage signal and said diploid signal; and
f) determining the copy number of said dosage region based on the ratio of said dosage signal to said diploid signal.

9. The method of claim 8, wherein said dosage probe mixture further comprises at least one dosage capture probe comprising a crosslinking agent, a label comprising a member of a specific binding pair, and a sequence that is substantially complementary to at least a portion of said dosage region and is distinct from the sequence of said at least one dosage reporter probe.

10. The method of claim 8 or 9, wherein said diploid probe mixture further comprises at least one diploid capture probe comprising a crosslinking agent, a label comprising a member of a specific binding pair, and a sequence that is substantially complementary to at least a portion of said diploid region and is distinct from the sequence of said at least one diploid reporter probe.

11. The method of claim 9, comprising the additional step of separating said crosslinked dosage probe: dosage region complex formed by said activating step using said at least one dosage capture probe.

12. The method of claim 10, comprising the additional step of separating said crosslinked diploid probe: diploid region complex formed by said activating step using said at least one diploid capture probe.

13. A method for determining the copy number of a dosage region in a sample, said method comprising:
a) hybridizing said dosage region to a dosage probe mixture, wherein said dosage probe mixture comprises a plurality of dosage probes comprising a crosslinking agent and having distinct sequences which are substantially complementary to a portion of said dosage region, said plurality of dosage probes further comprising:
i) at least one dosage reporter probe comprising a detectable label capable of producing a dosage signal; and
ii) at least one dosage capture probe comprising a label comprising a member of a specific binding pair;
b) activating said crosslinking agent to form a crosslinked dosage complex, whereby covalent crosslinks occur between said plurality of dosage probes and said dosage region when said dosage region is present in said sample;
c) separating said crosslinked dosage complex formed by said activating step using said member of a specific binding pair;
d) washing said crosslinked dosage complex at least once under high-stringency conditions;
e) detecting said dosage signal; and
f) determining the copy number of said dosage region based on the ratio of said dosage signal to a diploid signal.

14. The method of claim 13, comprising the additional steps of hybridizing a diploid probe mixture to a diploid region in said sample and performing said activating, separating, washing and detecting steps to obtain said diploid signal; wherein said diploid probe mixture comprises:
a) at least one diploid reporter probe comprising a sequence complementary to at least a portion of said diploid region, a crosslinking agent and a detectable label capable of producing said diploid signal, and
b) at least one diploid capture probe comprising a crosslinking agent, a label comprising a member of a specific binding pair, and a sequence which is substantially complementary to at least a portion of said diploid region and is distinct from the sequence of said at least one diploid reporter probe.

15. A method for determining the copy number of a dosage region in a sample, wherein said sample comprises at least one dosage region and at least one diploid region, said method comprising:
a) hybridizing said at least one dosage region to a dosage probe mixture to form a dosage hybridization complex, said dosage probe mixture comprising a plurality of dosage probes comprising a crosslinking agent and having distinct sequences substantially complementary to a portion of said dosage region, and wherein at least one of said plurality of dosage probes further comprises a detectable label capable of producing a dosage signal and at least one of said plurality of dosage probes further comprises a label comprising a member of a specific binding pair;
b) hybridizing said at least one diploid region to a diploid probe mixture to form a diploid hybridization complex, said diploid probe mixture comprising a plurality of diploid probes comprising a crosslinking agent and having distinct sequences substantially complementary to a portion of said diploid region, and wherein at least one of said plurality of diploid probes further comprises a detectable label capable of producing a diploid signal and at least one of said plurality of diploid probes further comprises a label comprising a member of a specific binding pair;
c) activating said crosslinking agent, whereby covalent crosslinks occur between said diploid probe mixture and said diploid region to form a crosslinked diploid complex, and between said dosage probe mixture and said dosage region to form a crosslinked dosage complex when said dosage region is present in said sample;
d) separating said crosslinked dosage complex and said crosslinked diploid complex formed by said activating step using said member of a specific binding pair;
e) washing said crosslinked dosage complex and said crosslinked diploid complex at least once under high-stringency conditions;
f) detecting said dosage signal and said diploid signal; and
g) determining the copy number of said dosage region based on the ratio of said dosage signal to said diploid signal.

16. A method for genotyping a target sequence in a sample, wherein said target sequence comprises a dosage region and an interrogation region comprising an interrogation position, said method comprising:
a) hybridizing said dosage region to a first crosslinkable probe mixture to form at least one first hybridization complex, said first crosslinkable probe mixture comprising at least one dosage reporter probe comprising a crosslinking agent, a detectable label capable of producing a dosage signal and a sequence substantially complementary to at least a portion of said dosage region;
b) hybridizing said interrogation region to a second crosslinkable probe mixture to form at least one second hybridization complex, said second crosslinkable probe mixture comprising at least one allele-specific detection probe comprising a crosslinking agent, a detectable label capable of producing an interrogation signal and a sequence substantially complementary to the sequence upstream and downstream of the interrogation position in said interrogation region;
c) activating said crosslinking agent, whereby said first hybridization complex becomes covalently crosslinked when said dosage region is present in said sample, and said second hybridization complex becomes covalently crosslinked when said detection position is perfectly complementary to said interrogation position;
d) washing said crosslinked first and second hybridization complexes at least once under high-stringency conditions; and
e) detecting said dosage signal to determine the copy number of said dosage region and detecting said interrogation signal to determine the identity of said interrogation position.

17. A method according to claim 16, wherein said second crosslinkable probe mixture comprises a plurality of allele-specific capture probes having distinct sequences that differ at said detection position, thereby enabling discrimination of alleles.
